SEQUENCE LISTING (103 sequences)

SEQ ID NO: 1 - DNA, 101 bp, Homo sapiens
cagggattgcccctcgtacttcttcgtattgtaggagtccaggtgcttaayaaaggtttcacgatgaggatgacgatggtatcaaggggctggatgtttaa

SEQ ID NO: 2 - DNA, 101 bp, Homo sapiens
gcgccatggggcaacccgggaacggcagcgccttcttgctggcacccaatrgaagccatgcgccggaccacgacgtcacgcaggaaagggacgaggtgtgg

SEQ ID NO: 3 - DNA, 101 bp, Homo sapiens
ggattgtgtcaggccttacctccttcttgcccattcagatgcactggtacmgggccacccaccaggaagccatcaactgctatgccaatgagacctgctgt

SEQ ID NO: 4 - DNA, 101 bp, Homo sapiens
ggctgtcttcacttccagggagggagggttatttactgcatgcttcatcaytgacgatctgcatggagagaacacaggaatcacattcactttgagccgct

SEQ ID NO: 5 - DNA, 101 bp, Homo sapiens
taagtaccaggcaagatttattaaatagaccagtggatttaccctaatgamctttttgaatttcgagggtaaagaaaaaaaagcattgtaagcatccagga

SEQ ID NO: 6 - DNA, 101 bp, Homo sapiens
tccctcaagcagtggaaagtcaatggtggtttttaaggaggtgaataatgygggctgaccgatgctttaagaagattaatacagcaatgtcttgaagagtg

SEQ ID NO: 7 - DNA, 101 bp, Homo sapiens
atcacaggagcctggtggaaatgaagtctttatgagggacacctgtattcrtctgtgcaggctgccataacaaaataccacagacagggtaacttaaacaa

SEQ ID NO: 8 - DNA, 101 bp, Homo sapiens
cataacctagaggctggataacgacatggacctcagatgaggagagaaaayggcatgaatggccattccaggggaagttataaggagaagaaaactgcatg

SEQ ID NO: 9 - DNA, 101 bp, Homo sapiens
ttgtagctgctcagtcagcactgtttgtgctgaattgaaacaatgagataygtacaaaggggctgtctctcactactggggatttaaagccaccccgaggc

SEQ ID NO: 10 - DNA, 101 bp, Homo sapiens
tcactcagagcagtctcaccctaaaaaaccttaggaaactggactgaggcyggaactgaccagtgaaggggaagtcaccacctttaatctgagagcaagca

SEQ ID NO: 11 - DNA, 101 bp, Homo sapiens
agttatatatgatttagtgaaggtgtcaaggcacgtgcataactcagatasatggcaggatgaaaaaggctgcagcttcgggaggaatgggctacaggact

SEQ ID NO: 12 - DNA, 101 bp, Homo sapiens
tttgttctcatgtcactctgtgggaattggggttcgagagctgacacaaaygctgatactctggctactgctattactgtgagtaacaaaccatcctttgt

SEQ ID NO: 13 - DNA, 101 bp, Homo sapiens
acaaataatttcccaaggaaatgagcagatcatggtgaaaaaaaaataacsaagcacacaagaaaacaaggcactatgagtaaaaaccagcagaaacagat

SEQ ID NO: 14 - DNA, 101 bp, Homo sapiens
tgattcaaacccagacttgcctttctctaaagcccatgcttttaaaagcaycacaatatctcctgaaggaaacaaggaaaaacagacaggatgcagatgga

SEQ ID NO: 15 - DNA, 101 bp, Homo sapiens
cctttccaagtaggagctgggatttgacagaagcagcaccagggagttgcygggatttctgaatgacatcactctagagggtcctatctcaggctttagcc

SEQ ID NO: 16 - DNA, 101 bp, Homo sapiens
tttcctagtgttccacaatccctaagttgttcaaatcaagtcttggaattktcatgaagttctctttccctttttcctctatatctgatccataaacaaat

SEQ ID NO: 17 - DNA, 101 bp, Homo sapiens
tgcacacacacgcattttcacatagatatttgtacaaatctaggttttttrtacacttccatatgattgcttaaataaatgtaggatgtagacatactcag

SEQ ID NO: 18 - DNA, 101 bp, Homo sapiens
ctcttatcacactgacttgtgtcatctcttgactgtcagtctctcccactraactacaaaccttctgagagcagaagccctttttcttttattgttttctc

SEQ ID NO: 19 - DNA, 101 bp, Homo sapiens
tggccctgtctctcactggtacaggagcctaggcaagttaatttaagtctygattttctcaacaatacattggggataatacccagttgggaaaaaagggc

SEQ ID NO: 20 - DNA, 101 bp, Homo sapiens
agctgggagggtgtgtctcagtgtctatggctgtggttcggtataagtctragcatgtctgccagggtgtatttgtgcctgtatgtgcgtgcctcggtggg

SEQ ID NO: 21 - DNA, 101 bp, Homo sapiens
ccttgcacaatcacccttccctctttgcccatggctgcacctccctccagycagattggagtttttgcagttccctctgcgagccttgtaacctctcttgt

SEQ ID NO: 22 - DNA, 101 bp, Homo sapiens
tgtggaagccctaaacgccaatgtggtattaggtaattaggtttagatgargtcatgaaagtgagccccaaggtgggattagtgcccttataagaagagga

SEQ ID NO: 23 - DNA, 101 bp, Homo sapiens
tttcactaccactgtgatcagcaaaactgggaaaagggcagaccttggctrtggcctctcacattgccccagtccatggcatgagcgtctcacccaaactt

SEQ ID NO: 24 - DNA, 101 bp, Homo sapiens
ctttggggtacacatccggtgctggggccagggaaagaatccaggttcccyaatcacacgaaaaaggtacctcctcctctgccaaagttcactggcaactc

SEQ ID NO: 25 - DNA, 101 bp, Homo sapiens
ggcacgtgcataactcagatacatggcaggatgaaaaaggctgcagcttcrggaggaatgggctacaggactcgtgcctatggtccatccaggttactgcc

SEQ ID NO: 26 - DNA, 101 bp, Homo sapiens
ttggctaatcccatttctagatcatgtttacttggcagatcatgccagtcrtctaattagcattcttcagggttattctacccccgcatggaccgctttgg

SEQ ID NO: 27 - DNA, 101 bp, Homo sapiens
ccctgcagcctcaaccataagaggcaggcacacctgggggagctgtttatragcgtatacctccaggcgtatattctctttcccagggatgttccatgctg

SEQ ID NO: 28 - DNA, 101 bp, Homo sapiens
gaggcaaatgcaagaatattctactggcaagtttcaaccgtctagacacartcaagattcctaggctaaacaattaagtggatattaggcccaaatggtaa

SEQ ID NO: 29 - DNA, 101 bp, Homo sapiens
ctctaagaatccagcccttgtcctacagttgggagctgtgggagatgcagmcttgttgatctgataggctaggatccctgataaggttggacactgccttt

SEQ ID NO: 30 - DNA, 101 bp, Homo sapiens
agacccatgcttcttcagaatcataaagtgtaagttattctactgaatgtsaagaaggcagtttgacttcctaacgagctgttctctgggacttttaattt

SEQ ID NO: 31 - DNA, 101 bp, Homo sapiens
aaaccaaaccaaaaaaacccaactttactaccaggcagattcttttctttsgctttagataatcgctatcaaaactttgtcatctcactagaatgttaatg

SEQ ID NO: 32 - DNA, 101 bp, Homo sapiens
taaaaagatataccacacagtgttaagaaaaacaggtaaattagatacaastctctgctatatactgaaagccatgggaacagggcgccaccgcctagaga

SEQ ID NO: 33 - DNA, 101 bp, Homo sapiens
aacacctccccaggatcttgtcctcaagtgtctaaacctcttgtactattyacatggtcctttgcggtctagtcctgcctctctctccagcctcatttctt

SEQ ID NO: 34 - DNA, 101 bp, Homo sapiens
tcagagcttgccaatagcgaattgttctgacccactagagccagccagagycagggcaaattttctgtaggcctctcatcaattaccatttattttccagg

SEQ ID NO: 35 - DNA, 101 bp, Homo sapiens
tgtgaaaagcgaggcccacgggaagccacgcaaacactgtgacacactgtsttgcaaaatctctcgtgacctagtggagtgaaaagtccaagttcatttta

SEQ ID NO: 36 - DNA, 101 bp, Homo sapiens
gttgtggagtggatgctttggcaagatggcggggagcggcgtccgccaagytacttctaccgccagcaccttcgtgaagcccattttcagtcgggacatga

SEQ ID NO: 37 - DNA, 101 bp, Homo sapiens
aaggcatagcctgggagtcaggcaattggtgttctagatccacctccgccyacaccgttagatgatctgaaataagtcacctcccctctctttgcctctgt

SEQ ID NO: 38 - DNA, 101 bp, Homo sapiens
ggctggcaatgcttttgccttttcactgcctgaactgagtccacaggaccrggtgctccagcccctcccactctggctccttcttggctcagccccctgaa

SEQ ID NO: 39 - DNA, 101 bp, Homo sapiens
taaataagtgtgaagatattgctcaggatctggctggcaatgcttttgccytttcactgcctgaactgagtccacaggaccaggtgctccagcccctccca

SEQ ID NO: 40 - DNA, 101 bp, Homo sapiens
tgtaaatccatgggaaagaaaagtttgttaacacactgctgaaaagtccakagtctgggttctagcccatctagccattagttgtgtgaccttggacaagc

SEQ ID NO: 41 - DNA, 101 bp, Homo sapiens
aatctctcgtgacctagtggagtgaaaagtccaagttcattttatccagcwtgaaaaagcccagtggtcagggacagtgacccactgtgctctggacagcc

SEQ ID NO: 42 - DNA, 101 bp, Homo sapiens
aagttgcactagactactctatttaagagctactttgattgaaagaacaarccaaataataatgatggtaattataacttatttccctcactaagttatat

SEQ ID NO: 43 - DNA, 101 bp, Homo sapiens
ccacacacttaaacaaaggaagcctacgagccttgtaagtggagggtcccratggggttaataagagattgacttccttatggggagtgtcgagcccagac

SEQ ID NO: 44 - DNA, 101 bp, Homo sapiens
atcctttcagttcagcactcttgggtcagtacgagtccaaagctattttckgatccttgatctgcctgaaatatgcaaataggaccttccctgggatatgt

SEQ ID NO: 45 - DNA, 101 bp, Homo sapiens
ctatttgcatatttcaggcagatcaaggatccgaaaatagctttggactcstactgacccaagagtgctgaactgaaaggattctacagtgttaaaatctt

SEQ ID NO: 46 - DNA, 101 bp, Homo sapiens
taagaatcgaatcattaactgagatcactcaagtagatcccttttccaacrcaatcaagagacaaaccaaaacacttagtctcctcttccatcctttacct

SEQ ID NO: 47 - DNA, 101 bp, Homo sapiens
ggcaatttctcaaagaacgaagtatgtgtgtggcaggcagtacttgacctstcttgcaaaataagccatactctttaaagagcatactactgttagggaga

SEQ ID NO: 48 - DNA, 101 bp, Homo sapiens
atgggaagaagggttgctagataggcaacaagaacatattcagtgcatcayctaattccacactgattcatattgatctgcttttacgtcttatctcccta

SEQ ID NO: 49 - DNA, 101 bp, Homo sapiens
tcaactttctctcagcctctcatccccacttctgatggattgatgttttawtctttccttcacctgacggaagacatggctgctggtggcacagcagttct

SEQ ID NO: 50 - DNA, 101 bp, Homo sapiens
aaaacaatgtgaaaataatagtgttcccaacttttatgtcatgttcatttmctccttcattccttccttcacgctatttatttcatgtggaaggctctccc

SEQ ID NO: 51 - DNA, 101 bp, Homo sapiens
taatgaaacccactgcatgtgcaggtattattttgttctcatgtcactctrtgggaattggggttcgagagctgacacaaatgctgatactctggctactg

SEQ ID NO: 52 - DNA, 101 bp, Homo sapiens
cgtcctgcaaggctctttgagcagcctttcaaggaggttccaaaagaggarttctcaaactattttaaccagccatagtattgtttaaaaaaaaacaaata

SEQ ID NO: 53 - DNA, 101 bp, Homo sapiens
caccacctgccagcctaacttgatggcccatttgggacttgaaagattgargccaactgggctgaattgagttaaaccccagttcaacaaaaatgctcagt

SEQ ID NO: 54 - DNA, 101 bp, Homo sapiens
ttcagcatttactctgctgcagatcagggtgtagaatcttctgaatgaggygtcatagagtctgatagcccagacatcagggcctagccccaacttgaaag

SEQ ID NO: 55 - DNA, 101 bp, Homo sapiens
actgatgatcaaagtatttgtatctatcaaagtcctggtgaaaaacaaacrtcatgctcaaatgaggtaatggaagaaagtttaatgaagagactgttgca

SEQ ID NO: 56 - DNA, 101 bp, Homo sapiens
atgaccatatcatcgtttttatttttccactatggtaacaaaaccctcagkcatgggttcacctgaccttgaattctgctgtatggatctgtgctgtccaa

SEQ ID NO: 57 - DNA, 101 bp, Homo sapiens
tcacagaggataaggcaacgatccatgaactcagtgaacaaaaatacaaakagtacctccgttgtgctgggtgctgggaacacagcagtgaacaaggtaaa

SEQ ID NO: 58 - DNA, 101 bp, Homo sapiens
gtaagtctatgcaaccacattggaaaactgattggcagtatcagtttactrctcaagctgaatgtatgcataccctgtgatactagatgtataacaaacag

SEQ ID NO: 59 - DNA, 98 bp, Homo sapiens
ataaagcaaatggaattattgagtgcatagaaagctgaggagagggtgtcrcagttttacatgtatatcatcaagagggttttttgtttgtttgtttg

SEQ ID NO: 60 - DNA, 101 bp, Homo sapiens
aactttaagggattctcccaaaagctaggacagagaagagacttgttttcrgtggcctcaagggggcagacttaagatccatgggtagaagtgagaaaatg

SEQ ID NO: 61 - DNA, 101 bp, Homo sapiens
actggttttgttcaagccttgcagattaccctccactactgtgcctaagayggctcttatccagcatggcacacacttctgctcatgctcacaatatttac

SEQ ID NO: 62 - DNA, 101 bp, Homo sapiens
aaaatataaaactttctctttattccatggcttctttctccactcatcatrgtgtttttactttgctatttatgtagctctaagaacaatttaaaattatc

SEQ ID NO: 63 - DNA, 101 bp, Homo sapiens
cagccctgaccgaggagtggcaggcctgaatcagttctcaccagaggttcrgagaaataggttgctctgatccccaatcaaatctattaggagttgtccca

SEQ ID NO: 64 - DNA, 101 bp, Homo sapiens
cagccaaggggcatgcccagaaggcatcttttaaatctattactgtaaacmactgatggttttttggaattttgctgagaatggtgtatgggttggggaca

SEQ ID NO: 65 - DNA, 101 bp, Homo sapiens
cttagcgctacatgcaagctatagtctcttctcatcttttcatttggttcraagggaaaaaggcaatcatcttacttttagggtagtcagaacctgtggaa

SEQ ID NO: 66 - DNA, 101 bp, Homo sapiens
ctttctaggaattgagagaattaattcctccgggtgttcccagaagggatrggagctcagatgctgcattaaggagaagaaatggtatgacttaagaggta

SEQ ID NO: 67 - DNA, 101 bp, Homo sapiens
ggtgatcctataaatatctgtggcttcaaccattacctcttaatactcccrtctatactgccagtctctacctcttctgtacttcaaattggtaaatttat

SEQ ID NO: 68 - DNA, 101 bp, Homo sapiens
aaaaaaaaaaaaaaagtgacgcggtcatttaactcagctgcaacttttcayggaaatgcaggaaagactaactcattgaatgatcagttgcctacttggaa

SEQ ID NO: 69 - DNA, 101 bp, Homo sapiens
aaatgtcctataataattatccattatccttttaaaaagaaaaaaatatgyttttaaaaataaaagcaatgcagtcagatgtgtggtgctgaagacttcct

SEQ ID NO: 70 - DNA, 101 bp, Homo sapiens
gctatcgacatactcataccacatggagactttctccttattaactgaaaragaattgaaaaagaatagtgttctcactctgaaattcactgttatttaac

SEQ ID NO: 71 - DNA, 101 bp, Homo sapiens
attttgaagaagggaagacaagtgcaaccggctgaggacggtgcacagagrccttctaaattcttctgcagtttgtagtgctacagataagtttttccatg

SEQ ID NO: 72 - DNA, 101 bp, Homo sapiens
tctggttggaggggaaaaagggaagaggagaggatgctgaagcctcacttkcttggccctccaactatgccagtcctggagaggaagaggagcttctgtag

SEQ ID NO: 73 - DNA, 101 bp, Homo sapiens
tgacaaaaaggagagggctatataaaaagctgtctgggaaaaacacatccyagagaaaggaagaggcaagacctgaggtggagcataagttcatgttctag

SEQ ID NO: 74 - DNA, 101 bp, Homo sapiens
cagtatttgccaaaatgttttctcagaacactagtgcccacatatgttctyggaaagaaaaacatggctctgtggccaagaaagatggatgaatgctacat

SEQ ID NO: 75 - DNA, 101 bp, Homo sapiens
actattggagtgacttgggggatttgacccatgggtgctaagtggtgtaayaggtaaaatgggagtgcagctgggcagcacttgctgcctggggaatgagg

SEQ ID NO: 76 - DNA, 101 bp, Homo sapiens
tcatgactcaggagttcaaatgggaaccctgaccagcacagatggtgccascacctcatctaaaccctgtggccaatttttcttttcttctgccttttccc

SEQ ID NO: 77 - DNA, 101 bp, Homo sapiens
cccatcaaattcaaatcaaaccccgcctcctatggacagcgtggctgcctycgtggacagccctgcttctctcctggggaagcacagagagggggcatgca

SEQ ID NO: 78 - DNA, 101 bp, Homo sapiens
catgttttaacggaaagtggcccagatacagagcttgaacctttcaccagmctttgtcactgtctcagaggcagggctcgctgacttgaggtggcaaactg

SEQ ID NO: 79 - DNA, 101 bp, Homo sapiens
ctgagtcaccctgcaagttccctgacaggagtttaacttcacatttcaackggttttccagtagagactcaagatatttgttgaattgaaagggatgcctg

SEQ ID NO: 80 - DNA, 101 bp, Homo sapiens
gccagtgaaatctcccttttcttgccctgtaggatactgaggaaagccacygacatttcgtggcacggctgacccgaaactatctttgtaactatagcgca

SEQ ID NO: 81 - DNA, 101 bp, Homo sapiens
cactactttcatttctctgctttcatgtattagggtgatcactgaactgcrggttttaacactggctgctgatcgccactccccaacactgagaaacagac

SEQ ID NO: 82 - DNA, 101 bp, Homo sapiens
gttcattggttgactgtctcagaatcactgattatcagggtgagaagagamcatcaggataatctaacccagtgaaacgctgatctgataatcacacaaat

SEQ ID NO: 83 - DNA, 101 bp, Homo sapiens
aaagaaaaaaaagaaaataggaggtgttctctacttcattaactatgtcasagagcaatttgaggctgtcacactccaaaagcatgacacacagtcctagg

SEQ ID NO: 84 - DNA, 101 bp, Homo sapiens
ctaactatgttactggttaagattcatttaaattcatttttctctaattaktaatgacaagaatgaaataatacctattacctttctcttatgtaaaatat

SEQ ID NO: 85 - DNA, 101 bp, Homo sapiens
ccacatcccaggaactgccacataagaaagggggaatccatgcatggactyaaagtactgaatcagcagctggaaatactaaagtaattcatgacccatag

SEQ ID NO: 86 - DNA, 101 bp, Homo sapiens
ttagagtgttgctggttaggggggaagtggacttggtcttgtaaaccaaayggtagagctaggcctaaatcatggacatatctgggaggcagatttggctc

SEQ ID NO: 87 - DNA, 101 bp, Homo sapiens
gattatcacccactttgaagccagcacatttctctcgttgctgtttcatgygaggaaccgtctaatacttatgttagtttctgttgccaccaggcaggtac

SEQ ID NO: 88 - DNA, 101 bp, Homo sapiens
agactaactattataacccagcaggcccaagacacaacaaagaaataaaayagtcaccaactatcggtgaacctgccccgatattcacgtaggttcttttc

SEQ ID NO: 89 - DNA, 101 bp, Homo sapiens
gacaagaagttgttctgtttgctctctgtgctggaagtcttgtaacaatcrtggcatgcgtgagattacagatgactcaagattgtgcagaaggaggcagc

SEQ ID NO: 90 - DNA, 101 bp, Homo sapiens
ctgggctctgttcctctgggcccccacccagcttaagagaaacagggccakccagcactgtgccttctgaaaaatcagtggtcagaattttctttctgtac

SEQ ID NO: 91 - DNA, 101 bp, Homo sapiens
atggtggccctttatacattacctgtatgcactcttggccttttgaggtgkcaggccctccccaagcagtcatcatgaatcatgatgggggtgtgaagggc

SEQ ID NO: 92 - DNA, 101 bp, Homo sapiens
actaatgttaaaagttcaccctagaggcaggtcaaggaaggtaattacaartaagcacacgaataaaagcctcagcaatgcgacccaccagtttataaaca

SEQ ID NO: 93 - DNA, 101 bp, Homo sapiens
atgaatgttgaattcctgaagctcagacttttttctctctggaagtttttrggttccactctgtcctcagtgttgtgaaatttcttgacaacaaaattggg

SEQ ID NO: 94 - DNA, 101 bp, Homo sapiens
aaatcaatcatgcaattaaaaattttcttctttgaacttctcaataacagygatctcaatctttttcatgtgagtttattttaggagatataaagccacag

SEQ ID NO: 95 - DNA, 101 bp, Homo sapiens
gaacgggggaaacaggagagggaagtgggaagagacccagcacaggtatgmgttcaggtgcaatcccagcctcagcagaccctaaaggggacccaggagta

SEQ ID NO: 96 - DNA, 101 bp, Homo sapiens
tctgtgctgggaacagcacttggtcttggggatacataggtgaacaagccrgactaaaattcttgccctcaggagattacattgcaggggaggagagaatg

SEQ ID NO: 97 - DNA, 101 bp, Homo sapiens
cagagtgagacttttgtttagacagagctgggttccttctcttcctattayctactatgtaacctgaggcaggctgcttaatttctttaaacttcagtttt

SEQ ID NO: 98 - DNA, 101 bp, Homo sapiens
ctctaaaagagccttctgtttcaccaagcaaagaggaaggaaaataaccamgtgtgggacattgagcaaagagattatttttaacaggaacagaaacccag

SEQ ID NO: 99 - DNA, 101 bp, Homo sapiens
aggaaaggattcatagtaaatctgaaaagtcaaaagtcaagcacatacagmaacacaagtgggctcataaaggggaagtgcaggagatgtctgtgggaaat

SEQ ID NO: 100 - DNA, 101 bp, Homo sapiens
tagaggagattatgcttttgacaagggcatgttacagagcagccattatcmgtggatttgcatgcatgactggttccatcattggtgttaccctcctacca

SEQ ID NO: 101 - DNA, 101 bp, Homo sapiens
tgtcttgagaaaaatacaactcaaaatggtcatattactcagatgatgccrgatgtctgatcctaaagacatttgaatctttattcttggtggtcaagtca

SEQ ID NO: 102 - DNA, 101 bp, Homo sapiens
ccacaacctggcaagcagtgtgcaaactccactatgattaatttataacaytgcatccaaaggggaaagactctcaggctgtattagggacttagagaaag

SEQ ID NO: 103 - DNA, 101 bp, Homo sapiens
atattctcagtatattcactgttttaggggaggaggtggaatgctataggkaagtatttgtcctgaatcattctgtggatacaccacacaaactcagaatt

(19) United States
(12) Patent Application Publication
(10) Pub. No.: US 2010/0317006 A1
(43) Pub. Date: Dec. 16, 2010

(54) SCA RISK STRATIFICATION BY PREDICTING PATIENT RESPONSE TO ANTI-ARRHYTHMICS

(75) Inventors: Orhan SOYKAN, Shoreview, MN (US); Tara NAHEY, Minneapolis, MN (US); Jeffrey LANDE, Minneapolis, MN (US)

Correspondence Address: Hahn & Voight-Medtronic, Inc. patent applications, 1012 14th Street, NW Suite 620, Washington, DC, 20005, US

(73) Assignee: MEDTRONIC, INC., Minneapolis, MN (US)

(21) Appl. No.: 12/778,603

(22) Filed: May 12, 2010

Related U.S. Application Data
(60) Provisional application No. 61/177,512, filed on May 12, 2009.

(51) Int. Cl. C12Q 1/68

(52) U.S. Cl. 435/6

(57) ABSTRACT

Genetic tests and methods for treatment based on markers to identify patients suffering from life-threatening ventricular tachy-arrhythmias, such as Ventricular Tachycardias (“VT”) and Ventricular Fibrillation (“VF”) that might lead to Sudden Cardiac Arrest (“SCA”) or Sudden Cardiac Death (“SCD”) are provided. Patients who cannot be sufficiently protected by medication alone, such as those refractory to anti-arrhythmic medication, are identified based on their genotype. The resulting information is used in a diagnostic test to identify and treat those patients who would benefit from the implantation of an Implantable Cardio Defibrillator (“ICD”).

### REFERENCE TO SEQUENCE LISTING

This application contains a “Sequence Listing” submitted as an electronic .txt file named MED_{—}10009_PROV_ST25, having a size of 24 kb, and created on Apr. 17, 2009. The information contained in the “Sequence Listing” is hereby incorporated by reference.

### BACKGROUND

Implantable Cardioverting Defibrillators (“ICDs”) effectively terminate life-threatening ventricular tachy-arrhythmias, such as Ventricular Tachycardias (“VT”) and Ventricular Fibrillation (“VF”) that might lead to Sudden Cardiac Arrest (“SCA”) or Sudden Cardiac Death (“SCD”). For many patients, ICDs are indicated for various cardiac related ailments, including myocardial infarction, ischemic heart disease, coronary artery disease, and heart failure. However, the use of these devices remains low, due in part to the lack of reliable markers indicating which patients are in need of these devices. Rather, it is more common that patients with various cardiac related ailments are prescribed anti-arrhythmic medications as the sole method of preventing SCA. Therefore, despite the demonstrated effectiveness of ICDs in SCA prevention, many patients who might benefit from an ICD do not receive one due to a lack of reliable methods for the identification of patients who cannot be sufficiently protected from SCA by medication alone.

### SUMMARY OF THE INVENTION

Methods for identifying patients who are refractory to β-blockers (or anti-arrhythmics), genetic tests and methods, including various DNA microarrays, through the use of the genetic markers, alone or in combination with other markers, to identify and distinguish such patients are provided. Diagnostic kits and methods for assessing the risk of Sudden Cardiac Arrest (“SCA”) and useful genetic markers are also provided. A method of assessing risk of SCA by predicting patient responses to anti-arrhythmics SNPs, genes, anti-arrhythmics in Class I-III are contemplated. The DNA microarrays can be in situ synthesized oligonucleotides, randomly or non-randomly assembled bead-based arrays, and mechanically assembled arrays of spotted material where the materials can be an oligonucleotide, a cNDA clone, or a Polymerase Chain Reaction (“PCR”) amplicon.

Specifically, an isolated nucleic acid molecule is provided having a Single Nucleotide Polymorphism (SNP) at position 51 selected from the group consisting of SEQ ID NO.'s 11-13, 19, 22-28, 30-32, 34-35, 37-55, 57, 61, 75-79, 83-88 and 102-103, or a complement thereof. A diagnostic kit for detecting one or more polymorphisms associated with no response to anti-arrhythmic medications (“Non-Responder-associated polymorphisms”) in a genetic sample having at least one probe for assessing the presence of a Single Nucleotide Polymorphism (“SNP”) in any one of SEQ ID NO.'s 11-13, 19, 22-28, 30-32, 34-35, 37-55, 57, 61, 75-79, 83-88 and 102-103 is provided. A diagnostic kit for detecting one or more polymorphisms in a genetic sample from a human subject refractory to Carvedilol having at least one probe for assessing the presence of a Single Nucleotide Polymorphism (SNP) at position 51 in any one of SEQ ID NO.'s 78, 77, 102, 61, 30, 19, 75, 76, 103, 31, 79, and 32, is provided. A diagnostic kit for detecting one or more polymorphisms in a genetic sample from a human subject refractory to Metoprolol having at least one probe for assessing the presence of a Single Nucleotide Polymorphism (SNP) at position 51 in any one of SEQ ID NO.'s 28, 22, 23, 24, 83, 11, 25, 26, 88, 27, 28, 55, 54, 53, 52, 87, 51, 12, 13, 50, 49, 48, 47, 57, 46, 45, 44, 43, 42, 35, 41, 34, 40, 39, 38 and 37 is provided. A diagnostic kit for detecting one or more polymorphisms in a genetic sample from a human subject refractory to Metoprolol having at least one probe for assessing the presence of a Single Nucleotide Polymorphism (SNP) at position 51 in any one of SEQ ID NO.'s 26, 22, 23, 24, 83, 11, 25, 26, 88, 27, 28, 55, 54, 53, 52, 87, 51, 12, 13, 50, 49, 48, 47, 57, 46, 45, 44, 43, 42, 35, 41, 34, 40, 39, 38 and 37 is provided. Using rs numbers, a diagnostic kit is provided for detecting one or more polymorphisms in a genetic sample from a human subject refractory to Carvedilol having at least one probe for assessing the presence of a Single Nucleotide Polymorphism (SNP) selected from the group consisting of rs5758637, rs5758627, rs9607885, rs2142695, rs17002868, rs12484402, rs5751239, rs5751240, rs9623538, rs17002872, rs5758645, and rs17002876. A diagnostic kit is provided for detecting one or more polymorphisms in a genetic sample from a human subject refractory to Metoprolol having at least one probe for assessing the presence of a Single Nucleotide Polymorphism (SNP) selected from the group consisting of rs151603, rs151591, rs151594, rs151595, rs6585252, rs11196566, rs151599, rs151600, rs7099933, rs151602, rs151603, rs180935, rs180934, rs180932, rs180929, rs7077623, rs180928, rs11196573, rs11196575, rs180925, rs180923, rs180922, rs180921, rs1860398, rs180919, rs180918, rs180917, rs180915, rs180914, rs17653278, rs180913, rs17574901, rs180912, rs180910, rs180909 and rs180908. A diagnostic kit is provided for detecting one or more polymorphisms in a genetic sample from a human subject refractory to Metoprolol having at least one probe for assessing the presence of a Single Nucleotide Polymorphism (SNP) selected from the group consisting of rs151600, rs151591, rs151594, rs151595, rs6585252, rs11196566, rs151599, rs151600, rs7099933, rs151602, rs151603, rs180935, rs180934, rs180932, rs180929, rs7077623, rs180928, rs11196573, rs11196575, rs180925, rs180923, rs180922, rs180921, rs1860398, rs180919, rs180918, rs180917, rs180915, rs180914, rs17653278, rs180913, rs17574901, rs180912, rs180910, rs180909 and rs180908.

Also provided is a DNA microarray for detecting one or more polymorphisms associated with no response to anti-arrhythmic medications (“Non-Responder-associated polymorphisms”) in a genetic sample made up of at least one probe for assessing the presence of a Single Nucleotide Polymorphism (“SNP”) in any one of the SEQ ID NO.'s 11-13, 19, 22-28, 30-32, 34-35, 37-55, 57, 61, 75-79, 83-88 and 102-103. Novel genetic markers for use in assessing response or non-response to anti-arrhythmic medications are provided. Methods of distinguishing patients having increased susceptibility to Sudden Cardiac Arrest (“SCA”) due to non-response to anti-arrhythmic medications, through the use of these markers, alone or in combination with other markers, are also provided. Further, methods of assessing the needs for an Implantable Cardio Defibrillator (“ICD”) in a patient are taught. Specifically, an isolated nucleic acid molecule is contemplated that is useful to predict SCA risk and the risk of non-response to anti-arrhythmic medication, and Single Nucleotide Polymorphisms (“SNPs”) selected from the group of SEQ ID NO.'s 11-13, 19, 22-28, 30-32, 34-35, 37-55, 57, 61, 75-79, 83-88 and 102-103 that can be used in the diagnosis, distinguishing, and detection thereof. A method of preventing SCA or SCD by implantation of an ICD in patients likely to have no response to anti-arrhythmic medications is also taught.

A method is provided for detecting one or more polymorphisms in a genetic sample obtaining a biological sample from the human subject, performing a hybridization to form a double-stranded nucleic acid between the nucleic acid sample and a probe using a DNA microarray and detecting the hybridization. Also provided is a method for analyzing a biological sample in a human subject by obtaining the biological sample from a human subject, hybridizing the biological sample with a probe to form a hybridization complex, and detecting said hybridization complex wherein the detection of a hybridization complex indicates a polymorphism or mutation associated with the human subject being refractory to anti-arrhythmics. Also provided is a method for analyzing a biological sample in a human subject by obtaining the biological sample from a human subject, transforming the biological sample with a probe to form a hybridization complex; and detecting said hybridization complex wherein the detection of a hybridization complex indicates a polymorphism or mutation associated with the human subject being refractory to anti-arrhythmics. Also provided is a method of determining the need for an Implantable Cardiac Defibrillator (“ICD”) in a human subject by obtaining the biological sample from a human subject, hybridizing the biological sample with a probe to form a hybridization complex, and a means for detecting said hybridization complex.

Provided are isolated nucleotides to be used in the diagnostic kits and methods that are useful to predict non-response to anti-arrhythmic medications, which are complementary to any one of SEQ ID NO.'s 11-13, 19, 22-28, 30-32, 34-35, 37-55, 57, 61, 75-79, 83-88 and 102-103 where the complement is between 3 and 101 nucleotides in length and overlaps a position 51 representing a SNP. An amplified nucleotide is further contemplated for use in the diagnostic kits containing a SNP embodied in any one of SEQ ID NO.'s 11-13, 19, 22-28, 30-32, 34-35, 37-55, 57, 61, 75-79, 83-88 and 102-103, or a complement thereof, overlapping position 51 wherein the amplified nucleotide is between 3 and 101 base pairs in length. A method of distinguishing patients is taught having no response to anti-arrhythmic medications from patients who do respond to anti-arrhythmic medications is provided, and genetic tests or methods thereof, where at least one SNP is detected at position 51 in any of the SEQ ID NO.'s 11-13, 19, 22-28, 30-32, 34-35, 37-55, 57, 61, 75-79, 83-88 and 102-103 in a nucleic acid sample from the patients. The presence or absence of the SNP can be used to assess whether the patient will respond to anti-arrhythmic medication.

A method of determining the risk of non-response to anti-arrhythmic medications in a patient, and a diagnostic kit thereof, is contemplated which requires identifying one or more SNPs at position 51 in any of SEQ ID NO.'s 11-13, 19, 22-28, 30-32, 34-35, 37-55, 57, 61, 75-79, 83-88 and 102-103 in a nucleic acid sample from the patient.

A method of detecting polymorphisms associated with a non-response to anti-arrhythmic medication (“Non-Response-associated polymorphisms”) and diagnostic kits or methods thereof, is further contemplated by extracting genetic material from a biological sample and screening the genetic material for at least one SNP in any of SEQ ID NO.'s 11-13, 19, 22-28, 30-32, 34-35, 37-55, 57, 61, 75-79, 83-88 and 102-103 at position 51.

A method for determining whether a patient needs an Implantable Cardio Defibrillator (“ICD”), and diagnostic kit thereof, is contemplated by identifying one or more SNPs at position 51 in any of SEQ ID NO.'s 11-13, 19, 22-28, 30-32, 34-35, 37-55, 57, 61, 75-79, 83-88 and 102-103 in a nucleic acid sample from the patient.

A method for the prevention of SCA or SCD by implantation of an ICD in patients with increased risk of having no response to anti-arrhythmic medication.

Those skilled in the art will recognize that the analysis of the nucleotides present in one or several of the SNP markers in a patient's nucleic acid can be done by any method or technique capable of determining nucleotides present at a polymorphic site. One of skill in the art would also know that the nucleotides present in the SNP markers can be determined from either nucleic acid strand or from both strands.

### BRIEF DESCRIPTION OF THE DRAWINGS

The foregoing and other features and aspects of the present disclosure will be best understood with reference to the following detailed description of a specific embodiment of the disclosure, when read in conjunction with the accompanying drawings.

FIG. 1 depicts an increase in the Number Needed to Treat (“NNT”) observed for the ICD therapy as the devices are implanted in patients with lower risks.

FIG. 2 depicts the Fast-Response Action Potential (e.g., ventricular myocyte) showing effective refractory period (ERP) and Ca⁺⁺, Na⁺ and K⁺ ion current where the Na⁺ channel blockers bind and block the fast sodium channels responsible for rapid depolarization (phase 0).

FIG. 3 depicts the simplified pathway of beta-adrenoceptor action.

FIG. 4 is an illustration of the analysis method used to determine the significance of a given SNP for patient response to anti-arrhythmic medication.

FIG. 5 is a mosaic plot of data for response to Carvedilol based on the patient genotype for rs5758637. The horizontal width of each block represents the prevalence of a given genotype in the study cohort. The vertical height of each block is proportional to the number of subjects in a given arm of the study.

FIG. 6 is a mosaic plot of data for response to Metoprolol based on the patient genotype for rs151603. The horizontal width of each block represents the prevalence of a given genotype in the study cohort. The vertical height of each block is proportional to the number of subjects in a given arm of the study.

FIG. 7 is a mosaic plot of data for response to Metoprolol based on the patient genotype for rs151600. The horizontal width of each block represents the prevalence of a given genotype in the study cohort. The vertical height of each block is proportional to the number of subjects in a given arm of the study.

FIG. 8 is a flow chart of the operation of the genetic test in conjunction with existing medical tests.

FIG. 9 is a list of rs numbers and corresponding SEQ ID NO.'s containing chromosome, coordinate, band, position, and other gene and population information.

### DETAILED DESCRIPTION OF THE INVENTION

The invention relates to diagnostic kits and methods using a nucleic acid molecule that can predict the risk for Sudden Cardiac Arrest (“SCA”) or Sudden Cardiac Death (“SCD”) due to non-response to anti-arrhythmic medication, having a single nucleotide polymorphism (“SNP”) selected from the group of SEQ ID NO.'s 11-13, 19, 22-28, 30-32, 34-35, 37-55, 57, 61, 75-79, 83-88 and 102-103, and methods for diagnosing and distinguishing human subjects (patients) for implanting an Implantable Cardiac Defibrillator (“ICD”) in a patient in need thereof.

Unless otherwise defined, all technical and scientific terms used herein have the same meaning as commonly understood by one of ordinary skill in the art to which this invention belongs. For the purposes of the present invention, the following terms are defined below:

The terms “a,” “an,” and “the” include the plural referents unless the context clearly dictates otherwise.

The term “isolated” refers to nucleic acid, or a fragment thereof, that has been removed from its natural cellular environment.

The term “nucleic acid” refers to a deoxyribonucleotide or ribonucleotide polymer in either single- or double-stranded form and, unless otherwise limited, encompasses known analogues of natural nucleotides that hybridize to nucleic acids in a manner similar to naturally occurring nucleotides. The term “nucleic acid” encompasses the terms “oligonucleotide” and “polynucleotide.”

“Probes” or “primers” refer to single-stranded nucleic acid sequences that are complementary to a desired target nucleic acid. The 5′ and 3′ regions flanking the target complement sequence reversibly interact by means of either complementary nucleic acid sequences or by attached members of another affinity pair. Hybridization can occur in a base-specific manner where the primer or probe sequence is not required to be perfectly complementary to all of the sequences of a template. Hence, non-complementary bases or modified bases can be interspersed into the primer or probe, provided that base substitutions do not inhibit hybridization. The nucleic acid template may also include “nonspecific priming sequences” or “nonspecific sequences” to which the primers or probes have varying degrees of complementarity. In certain embodiments, a probe or primer comprises 101 or fewer nucleotides, from about 3 to 101 nucleotides, from about 5 to 85, from about 6 to 75, from about 7 to 60, from about 8 to 50, from about 10 to 45, from about 12 to 30, from about 12 to 25, from about 15 to 20, or from about any number of base pairs flanking the 5′ and 3′ side of a region of interest to sufficiently identify, or result in hybridization. Further, the ranges can be chosen from group A and B where for A: the probe or primer is greater than 5, greater than 10, greater than 15, greater than 20, greater than 25, greater than 30, greater than 40, greater than 50, greater than 60, greater than 70, greater than 80, greater than 90 and greater than 100 base pairs in length. For B, the probe or primer is less than 102, less than 95, less than 90, less than 85, less than 80, less than 75, less than 70, less than 65, less than 60, less than 55, less than 50, less than 45, less than 40, less than 35, less than 30, less than 25, less than 20, less than 15, or less than 10 base pairs in length. In other embodiments, the probe or primer is at least 70% identical to the contiguous nucleic acid sequence or to the complement of the contiguous nucleotide sequence, for example, at least 80% identical, at least 90% identical, at least 95% identical, and is capable of selectively hybridizing to the contiguous nucleic acid sequence or to the complement of the contiguous nucleotide sequence. Preferred primer lengths include 25 to 35, 18 to 30, and 17 to 24 nucleotides. Often, the probe or primer further comprises a label, e.g., a radioisotope, fluorescent compound, enzyme, or enzyme co-factor.

To obtain high quality primes, primer length, melting temperature (Tₘ), GC content, specificity, and intra- or inter-primer homology are taken into account in the present invention. You et al., “BatchPrimer3: A high throughput web application for PCR and sequencing primer design,” BMC Bioinformatics 2008; 9:253; Yang, X. et al., “Recent developments in primer design for DNA polymorphism and mRNA profiling in higher plants”, Plant Methods 2006; 2 (1):4. Primer specificity is related to primer length and the final 8 to 10 bases of the 3′ end sequence where a primer length of 18 to 30 bases is one possible embodiment. Abd-Elsalam K. A., “Bioinformatics tools and guideline for PCR primer design,” Africa J. of Biotechnol. 2003; 2 (5):91-95. Tₘ is closely correlated to primer length, GC content and primer base composition. One preferred primer Tₘ is in the range of 50 to 65° C. with GC content in the range of 40 to 60% for standard primer pairs. Dieffenbatch, C. W. et al, “General concepts for PCR primer design,” In PCR primer, A Laboratory Manual. Edited by: Dieffenbatch C W, Dveksler G S. New York, Cold Spring Harbor Laboratory Press; 1995:133-155. However, an optimal primer length varies depending on different types of primers. For example, SNP genotyping primers may require a longer primer length of 25 to 35 bases to enhance their specificity, and thus the corresponding Tₘ might be higher than 65° C. Also, a suitable Tₘ can be obtained by setting a broader GC content range (20 to 80%).

The probes or primers can also be variously referred to as antisense nucleic acid molecules, polynucleotides or oligonucleotides, and can be constructed using chemical synthesis and enzymatic ligation reactions known in the art. For example, an antisense nucleic acid molecule (e.g. an antisense oligonucleotide) can be chemically synthesized using naturally occurring nucleotides or variously modified nucleotides designed to increase the biological stability of the molecules or to increase the physical stability of the duplex formed between the antisense and sense nucleic acids. The primers or probes can further be used in Polymerase Chain Reaction (“PCR”) amplification.

The term “genetic material” refers to a nucleic acid sequence that is sought to be obtained from any number of sources, including without limitation, whole blood, a tissue biopsy, lymph, bone marrow, hair, skin, saliva, buccal swabs, purified samples generally, cultured cells, and lysed cells, and can comprise any number of different compositional components (e.g. DNA, RNA, tRNA, siRNA, mRNA, or various non-coding RNAs). The nucleic acid can be isolated from samples using any of a variety of procedures known in the art. In general, the target nucleic acid will be single stranded, though in some embodiments the nucleic acid can be double stranded, and a single strand can result from denaturation. It will be appreciated that either strand of a double-stranded molecule can serve as a target nucleic acid to be obtained. The nucleic acid sequence can be methylated, non-methylated, or both, and can contain any number of modifications. Further, the nucleic acid sequence can refer to amplification products as well as to the native sequences.

Hybridization is the ability of two nucleotide sequences to bind with each other based on a degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like. Stringent conditions are conditions under which a probe can hybridize to its target subsequence, but to no other sequences. Stringent conditions are sequence-dependent and are different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide or tetraalkyl ammonium salts. For example, conditions of SxSSPE (750 mM NaCl, 50 mM Na Phosphate, 5 mM EDTA, pH 7.4) and a temperature of 25-30° C. are suitable for allele-specific probe hybridizations. Sambrook et al. Molecular Cloning 1989.

Allele Specific Oligomer (“ASO”) refers to a primary oligonucleotide having a target specific portion and a target-identifying portion, which can query the identity of an allele at a SNP locus. The target specific portion of the ASO of a primary group can hybridize adjacent to the target specific portion and can be made by methods well known to those of ordinary skill The ordinary meaning of the term “allele” is one of two or more alternate forms of a gene occupying the same locus in a particular chromosome or linkage structure and differing from other alleles of the locus at one or more mutational sites. Rieger et al., Glossary of Genetics, 5th Ed. (Springer-Verlag, Berlin 1991), p. 16.

Bi-allelic and multi-allelic refers to two, or more than two alternate forms of a SNP, respectively, occupying the same locus in a particular chromosome or linkage structure and differing from other alleles of the locus at a polymorphic site.

### DNA Microarrays

Numerous forms of diagnostic kits employing arrays of nucleotides are known in the art. They can be fabricated by any number of known methods including photolithography, pipette, drop-touch, piezoelectric, spotting and electric procedures. The DNA microarrays generally have probes that are supported by a substrate so that a target sample is bound or hybridized with the probes. In use, the microarray surface is contacted with one or more target samples under conditions that promote specific, high-affinity binding of the target to one or more of the probes. A sample solution containing the target sample typically contains radioactively, chemoluminescently or fluorescently labeled molecules that are detectable. The hybridized targets and probes can also be detected by voltage, current, or electronic means known in the art.

Optionally, a plurality of microarrays may be formed on a larger array substrate. The substrate can be diced into a plurality of individual microarray dies in order to optimize use of the substrate. Possible substrate materials include siliceous compositions where a siliceous substrate is generally defined as any material largely comprised of silicon dioxide. Natural or synthetic assemblies can also be employed. The substrate can be hydrophobic or hydrophilic or capable of being rendered hydrophobic or hydrophilic and includes inorganic powders such as silica, magnesium sulfate, and alumina; natural polymeric materials, particularly cellulosic materials and materials derived from cellulose, such as fiber-containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), etc.; either used by themselves or in conjunction with other materials; glass available as Bioglass, ceramics, metals, and the like. The surface of the substrate is then chemically prepared or derivatized to enable or facilitate the attachment of the molecular species to the surface of the array substrate. Surface derivatizations can differ for immobilization of prepared biological material, such as cDNA, and in situ synthesis of the biological material on the microarray substrate. Surface treatment or derivatization techniques are well known in the art. The surface of the substrate can have any number of shapes, such as strip, plate, disk, rod, particle, including bead, and the like. In modifying siliceous or metal oxide surfaces, one technique that has been used is derivatization with bifunctional silanes, i.e., silanes having a first functional group enabling covalent binding to the surface and a second functional group that can impart the desired chemical and/or physical modifications to the surface to covalently or non-covalently attach ligands and/or the polymers or monomers for the biological probe array. Adsorbed polymer surfaces are used on siliceous substrates for attaching nucleic acids, for example cDNA, to the substrate surface. Since a microarray die may be quite small and difficult to handle for processing, an individual microarray die can also be packaged for further handling and processing. For example, the microarray may be processed by subjecting the microarray to a hybridization assay while retained in a package.

Various techniques can be employed for preparing an oligonucleotide for use in a microarray. In situ synthesis of oligonucleotide or polynucleotide probes on a substrate is performed in accordance with well-known chemical processes, such as sequential addition of nucleotide phosphoramidites to surface-linked hydroxyl groups. Indirect synthesis may also be performed in accordance with biosynthetic techniques such as Polymerase Chain Reaction (“PCR”). Other methods of oligonucleotide synthesis include phosphotriester and phosphodiester methods and synthesis on a support, as well as phosphoramidate techniques. Chemical synthesis via a photolithographic method of spatially addressable arrays of oligonucleotides bound to a substrate made of glass can also be employed. The probes or oligonucleotides, themselves, can be obtained by biological synthesis or by chemical synthesis. Chemical synthesis provides a convenient way of incorporating low molecular weight compounds and/or modified bases during specific synthesis steps. Furthermore, chemical synthesis is very flexible in the choice of length and region of target polynucleotides binding sequence. The oligonucleotide can be synthesized by standard methods such as those used in commercial automated nucleic acid synthesizers.

Immobilization of probes or oligonucleotides on a substrate or surface may be accomplished by well-known techniques. One type of technology makes use of a bead-array of randomly or non-randomly arranged beads. A specific oligonucleotide or probe sequence is assigned to each bead type, which is replicated any number of times on an array. A series of decoding hybridizations is then used to identify each bead on the array. The concept of these assays is very similar to that of DNA chip based assays. However, oligonucleotides are attached to small microspheres rather than to a fixed surface of DNA chips. Bead-based systems can be combined with most of the allele-discrimination chemistry used in DNA chip based array assays, such as single-base extension and oligonucleotide ligation assays. The bead-based format has flexibility for multiplexing and SNP combination. In bead-based assays, the identity of each bead needs is determined where that information is combined with the genotype signal from the bead to assign a “genotype call” to each SNP and individual.

One bead-based genotyping technology uses fluorescently coded microspheres developed by Luminex. Fulton R., et al, “Advanced multiplexed analysis with the FlowMetrix system,” Clin. Chem. 1997; 43: 1749-56. These beads are coated with two different dyes (red and orange), and can be identified and separated using flow cytometry, based on the amount of these two dyes on the surface. By having a hundred types of microspheres with a different red:orange signal ratio, a hundred-plex detection reaction can be performed in a single tube. After the reaction, these microspheres are distinguished using a flow fluorimeter where a genotyping signal (green) from each group of microspheres is measured separately. This bead-based platform is useful in allele-specific hybridization, single-base extension, allele-specific primer extension, and oligonucleotide ligation assay. In a different bead-based platform commercialized by Illumina, microspheres are captured in solid wells created from optical fibers. Michael K et al., “Randomly ordered addressable high-density optical sensor arrays,” Anal. Chem. 1998; 70: 1242-48.' Steemers F. et al., “Screening unlabeled DNA targets with randomly ordered fiber-optic gene arrays,” Nat. Biotechnol. 2000; 18: 91-94. The diameter of each well is similar to that of the spheres, allowing only a single sphere to fit in one well. Once the microspheres are set in these wells, all of the spheres can be treated like a high-density microarray. The high degree of replication in DNA microarray technology makes robust measurements for each bead type possible. Bead-array technology is particularly useful in SNP genotyping. Software used to process raw data from a DNA microarray or chip is well known in the art and employs various known methods for image processing, background correction and normalization. Many available public and proprietary software packages are available for such processing whereby a quality assessment of the raw data can be carried out, and the data then summarized and stored in a format which can be used by other software to perform additional analyses.

Hybridization probes can be labeled with a radioactive substance for easy detection. Grunstein et al., Proc. Natl. Acad. Sci. USA 72:3961 (1975) and Southern, J. Mol. Biol. 98:503 (1975) describe hybridization techniques using radio-labeled nucleic acid probes. Advantageously, nucleic acid hybridization probes can have high sensitivity and specificity. Radioactive labels can be detected with a phosphor imager or autoradiography film. Radioactive labels are most often used with nylon membrane macro-arrays. Suitable radioactive labels can be, for example, but not limited to isotopes like ¹²⁵I or ³²P. The detection of radioactive labels is, for example, performed by the placement of medical X-ray film directly against the substrate which develops as it is exposed to the label, which creates dark regions which correspond to the emplacement of the probes of interest.

Known methods of electrically detecting hybridization can be used such as electrochemical impedance spectroscopy. This technique can be used to investigate the changes in interfacial electrical properties that arise when DNA-modified Si(111) surfaces are exposed to solution-phase DNA oligonucleotides with complementary and non-complementary sequences. The n- and p-type silicon(111) samples can be covalently linked to DNA molecules via direct Si—C linkages without any intervening oxide layer. Exposure to solutions containing DNA oligonucleotides with the complementary sequence can produce significant changes in both the real and imaginary components of electrical impedance, while exposure to DNA with non-complementary sequences generate negligible responses. These changes in electrical properties can be corroborated with fluorescence measurements and reproduced in multiple hybridization-denaturation cycles. Additionally, the ability to detect DNA hybridization is strongly frequency-dependent wherein modeling of the response and comparison of results on different silicon bulk doping shows that the sensitivity to DNA hybridization arises from DNA-induced changes in the resistance of the silicon substrate and the resistance of the molecular layers. Wei et al., “Direct electrical detection of hybridization at DNA-modified silicon surfaces”, Biosensors and Bioelectronics 2004 Apr. 15; 19 (9):1013-9. Also, macroporous silicon can be used as an electrical sensor for real time, label free detection of DNA hybridization whereby electrical contact is made exclusively on a back side of a substrate to allow complete exposure of a porous layer to DNA. Hybridization of a DNA probe with its complementary sequence produces a reduction in the impedance and a shift in the phase angle resulting from a change in dielectric constant inside the porous matrix and a modification of a depletion layer width in the crystalline silicon structure. Again, the effect of the DNA charge on the response can be corroborated using peptide nucleic acid (PNA), which is an uncharged analog of DNA. Single Nucleotide Polymorphism (“SNP”)

Generally, genetic variations are associated with human phenotypic diversity and sometimes disease susceptibility. As a result, variations in genes may prove useful as markers for disease or other disorder or condition. Variation at a particular genomic location is due to a mutation event in the conserved human genome sequence, leading to two possible nucleotide variants at that genetic locus. If both nucleotide variants are found in at least 1% of the population, that location is defined as a Single Nucleotide Polymorphism (“SNP”). Moreover, SNPs in close proximity to one another are often inherited together in blocks called haplotypes. One phenomenon of SNPs is that they can undergo linkage disequilibrium, which refers to the tendency of specific alleles at different genomic locations to occur together more frequently than would be expected by random change. Alleles at given loci are said to be in complete equilibrium if the frequency of any particular set of alleles (or haplotype) is the product of their individual population frequencies. Several statistical measures can be used to quantify this relationship. Devlin and Risch 1995 Sep. 20; 29 (2):311-22).

With respect to alleles, a more common nucleotide is known as the major allele and the less common nucleotide is known as the minor allele. An allele found to have a higher than expected prevalence among individuals positive for a given outcome is considered a risk allele for that outcome. An allele found to have a lower than expected prevalence among individuals positive for an outcome is considered a protective allele for that outcome. But while the human genome harbors 10 million “common” SNPs, minor alleles indicative of heart disease are often only shared by as little as one percent of a population.

Hence, as provided herein, certain SNPs found by one or a combination of these methods have been useful as genetic markers for risk-stratification of SCD or SCA in individuals. Further, certain other SNPs found by one or combinations of these methods are useful as genetic markers for patient response to anti-arrhythmic medications. Genome-wide association studies are used to identify disease susceptibility genes for common diseases and involve scanning thousands of samples, either as case-control cohorts or in family trios, utilizing hundreds of thousands of SNP markers located throughout the human genome. Algorithms can then be applied that compare the frequencies of single SNP alleles, genotypes, or multi-marker haplotypes between disease and control cohorts. Regions (loci) with statistically significant differences in allele or genotype frequencies between cases and controls, pointing to their role in disease, are then analyzed. For example, following the completion of a whole genome analysis of patient samples, SNPs for use as clinical markers can be identified by any, or combination, of the following three methods:

(1) Statistical SNP Selection Method: Univariate or multivariate analysis of the data is carried out to determine the correlation between the SNPs and the study outcome, non-response to anti-arrhythmic medications for the present invention. SNPs that yield low-p values are considered as markers. These techniques can be expanded by the use of other statistical methods such as linear regression.

(2) Logical SNP Selection Method: Clustering algorithms are used to segregate the SNP markers into categories which would ultimately correlate with the patient outcomes. Classification and Regression Tree (“CART”) is one of the clustering algorithms that can be used. In that case, SNPs forming the branching nodes of the tree will be the markers of interest.

(3) Biological SNP Selection Method: SNP markers are chosen based on the biological effect of the SNP, as it might affect the function of various proteins. For example, a SNP located on a transcribed or a regulatory portion of a gene that is involved in ion channel formation would be good candidates. Similarly, a group of SNPs that are shown to be located closely on the genome would also hint the importance of the region and would constitute a set of markers.

Genetic markers are non-invasive, cost-effective and conducive to mass screening of individuals. The SNPs identified herein can be effectively used alone or in combination with other SNPs as well as with other clinical markers for risk-stratification, assessment, and diagnosis of non-response to anti-arrhythmic medications. Further, these genetic markers in combination with other clinical markers for SCA are effectively used for identification and implantation of ICDs in individuals who are at risk of not responding to anti-arrhythmic medications. The genetic markers taught herein provide greater specificity and sensitivity in identification of individuals at risk for SCA or SCD due to non-response to anti-arrhythmic medications.

### Sudden Cardiac Arrest (“SCA”)

Sudden Cardiac Arrest (“SCA”), also known as, Sudden Cardiac Death (“SCD”) results from an abrupt loss of heart function. It is commonly brought on by an abnormal heart rhythm. SCD occurs within a short time period, which is generally less than an hour from the onset of symptoms. Despite recent progress in the management of cardiovascular disorders generally, and cardiac arrhythmias in particular, SCA remains a problem for the practicing clinician as well as a major public health issue.

In the United States, SCA accounts for approximately 325,000 deaths per year. More deaths are attributable to SCA than to lung cancer, breast cancer, or AIDS. This represents an incidence of 0.1-0.2% per year in the adult population. Myerburg, R J et al., “Cardiac arrest and sudden cardiac death,” In Braunwald E, ed.: A Textbook of Cardiovascular Medicine. 6^{th} ed. Philadelphia: Saunders; W B., 2001: 890-931 and American Cancer Society. Cancer Facts and Fig.s 2003: 4, Center for Disease Control 2004.

In 60% to 80% of cases, SCA occurs in the setting of Coronary Artery Disease (“CAD”). Most instances involve Ventricular Tachycardias (“VT”) degenerating to Ventricular Fibrillation (“VF”) and subsequent asystole. Fibrillation occurs when transient neural triggers impinge upon an unstable heart causing normally organized electrical activity in the heart to become disorganized and chaotic. Complete cardiac dysfunction results. Non-ischemic cardiomyopathy and infiltrative, inflammatory, and acquired valvular diseases account for most other SCA, or SCD, events. A small percentage of SCAs occur in the setting of ion channel mutations responsible for inherited abnormalities such as the long/short QT syndromes, Brugada syndrome, and catecholaminergic ventricular tachycardia. These conditions account for a small number of SCAs. In addition, other genetic abnormalities such as hypertrophic cardiomyopathy and congenital heart defects such as anomalous coronary arteries are responsible for SCA.

Currently, five arrhythmia markers are often used for risk assessment in Myocardial Infarction (“MI”) patients: (1) Heart Rate (“HR”) Variability, (2) severe ventricular arrhythmia, (3) signal averaged Electro Cardio Gram (“ECG”), (4) left ventricular Ejection Fraction (“EF”) and (5) electrophysiology (“EP”) (studies). Table 1 illustrates the mean sensitivity and specificity values for each of these five arrhythmia markers for MI patients. As shown, these markers have relatively high specificity values, but low sensitivity values.

TABLE 1

| | | Severe | | Left | |
|---|---|---|---|---|---|
| | HR | Ventricular | Signal | Ventricular | |
| | Variability | Arrhythmia | Averaged | Ejection | Electrophysiology |
| Test | on AECG | on AECG | ECG | Fraction (EF) | (EP) Studies |
| Sensitivity | 49.8% | 42.8% | 62.4% | 59.1% | 61.8% |
| Specificity | 85.8% | 81.2% | 77.4% | 77.8% | 84.1% |

The most commonly used marker, EF, has a sensitivity of 59%, meaning that 41% of the patients would be missed if EF were the only marker used. Although EP studies provide slightly better indications, they are not performed very frequently due to their rather invasive nature. Hence, the identification of patients who have a propensity toward SCA remains as an unmet medical need. Furthermore, it is anticipated that this need would increase over time as the implantable cardioverting defibrillators (“ICDs”) are implanted in patients who are in lower risk categories.

ECG parameters indicative of SCA or SCD are QRS duration, late potentials, QT dispersion, T-wave morphology, Heart rate variability, and T-wave alternans. Electrical alternans is a pattern of variation in the shape of the ECG waveform that appears on an every-other-beat basis. In humans, alternation in ventricular repolarization, namely, repolarization alternans, has been associated with increased vulnerability to ventricular tachycardia/ventricular fibrillation and sudden cardiac death. Pham, Q., et al., “T-wave alternans: marker, mechanism, and methodology for predicting sudden cardiac death. Journal of Electrocardiology”, 36: 75-81. Analysis of the morphology of an ECG (i.e., T-wave alternans and QT interval dispersion) has been recognized as means for assessing cardiac vulnerability.

Certain biological factors are predictive of risk for SCA, such as a previous clinical event, ambient arrhythmias, cardiac response to direct stimulations, and patient demographics. Similarly, analysis of heart rate variability has been proposed as a means for assessing autonomic nervous system activity, the neural basis for cardiac vulnerability. Heart rate variability, a measure of beat-to-beat variations of sinus-initiated RR intervals, with its Fourier transform-derived parameters, is blunted in patients at risk for SCD. Bigger, J T. “Heart rate variability and sudden cardiac death”, In: Zipes D P, Jalife J, eds. Cardiac Electrophysiology: From Cell to Bedside. Philadelphia, Pa.: W B Saunders; 1999.

Patient history is helpful to analyze the risk of SCA or SCD. For example, in patients with ventricular tachycardia after myocardial infarction, on the basis of clinical history, the following four variables identify patients at increased risk of sudden cardiac death: (1) syncope at the time of the first documented episode of arrhythmia, (2) New York Heart Association (“NYHA”) Classification class III or IV, (3) ventricular tachycardia/fibrillation occurring early after myocardial infarction (3 days to 2 months), and (4) history of previous myocardial infarctions. Unfortunately, most of these clinical indicators lack sufficient sensitivity, specificity, and predictive accuracy to pinpoint the patient at risk for SCA, with a degree of accuracy that would permit using a specific therapeutic intervention before an actual event.

For example, the disadvantage of focusing solely on ejection fraction is that many patients whose ejection fractions exceed commonly used cut offs still experience sudden death or cardiac arrest. Because EF is not specific in predicting mode of death, decision-making for the implantation of an ICD solely on EF will not be optimal. Buxton, A E et al., “Risk stratification for sudden death: do we need anything more than ejection fraction?” Card. Electrophysiology Rev. 2003; 7: 434-7. Although, electrophysiological (“EP”) studies provide slightly better indication, they are not performed very frequently due to their invasive nature and high cost.

Conventional methods for assessing vulnerability to SCA or SCD often rely on power spectral analysis (Fourier analysis) of the cardiac electrogram. However, the power spectrum lacks the ability to track many of the rapid arrhythmogenic changes which characterize T-wave alternans, dispersions and heart rate variability. As a result, a non-invasive diagnostic method of predicting vulnerability to SCA or SCD by the analysis of ECG has not achieved widespread clinical acceptance.

Similarly, both baroflex sensitivity and heart rate variability judge autonomic modulation at the sinus node, which is taken as a surrogate for autonomic actions at the ventricular level. Autonomic effects at the sinus node and ventricle can easily be dissociated experimentally and may possibly be a cause of false-positive or false-negative test results. Zipes, D P et al., “Sudden Cardiac Death”; Circulation. 1998;98:2334-2351.

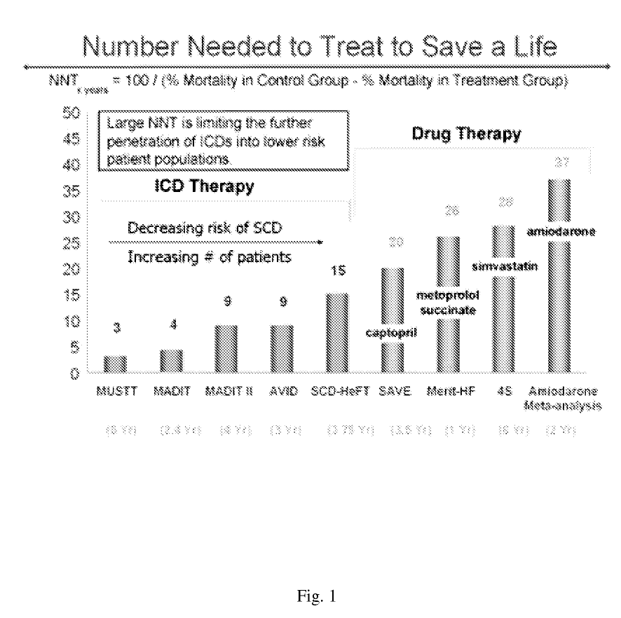

As shown in FIG. 1, an increase in the Number Needed to Treat (“NNT”) has been observed for the ICD therapy as the devices are implanted in patients with lower risks. NNT is an epidemiological measure used in assessing the effectiveness of a health-care intervention. The NNT is the number of patients who need to be treated in order to prevent a single negative outcome. Currently, in the case of ICDs, devices must be implanted in approximately 17 patients to prevent one death. The other 16 patients may not experience a life threatening arrhythmia and may not receive a treatment. Reduction of the NNT for ICDs would yield to better patient identification methods and allow delivery of therapies to individuals who need them. As a result, it is believed that the need for risk stratification of patients might increase over time as the ICDs are implanted in patients who are generally considered to be at lower risk categories. The net result of the lack of more specific markers for both life threatening arrhythmias and non-response to anti-arrhythmic medications is the presence of a population of patients who would benefit from ICD therapy but who are not currently indicated.

### Anti-Arrhythmic Medications

Currently, patients who are believed to be susceptible to SCA are treated with anti-arrhythmic medications. Many of these patients are also candidates for an ICD implant. However, since the NNT for an ICD is considered to be higher than desired, many patients do not receive these lifesaving devices as shown in FIG. 1. Because the anti-arrhythmic medications do not prevent everyone from a SCA, many patients who are taking the medication are in fact left without any protection against SCA. According to some estimates, up to 40% of patients do not respond to anti-arrhythmic drugs. Spear, Brian B. et al., “Clinical Application of Pharmacogenetics,” TRENDS in Molecular Medicine, 2001; 7 (5) 201-204.

Anti-arrhythmic drugs modify the cellular electrophysiology of the cardiomyocytes by acting on the molecular pathways governing the formation of the action potential. They can be grouped into four basic classes as shown in Table 2.

TABLE 2

| Class | Basic Mechanism | Comments |
|---|---|---|
| I | sodium-channel | Reduce phase 0 slope and peak of action |
| | blockade | potential. |
| IA | moderate | Moderate reduction in phase 0 slope; increase |
| | | APD; increase ERP. |
| IB | weak | Small reduction in phase 0 slope; reduce APD; |
| | | decrease ERP. |
| IC | strong | Pronounced reduction in phase 0 slope; no |
| | | effect on APD or ERP. |
| II | beta-blockade | Block sympathetic activity; reduce rate and |
| | | conduction. |
| III | potassium- | Delay repolarization (phase 3) and thereby |
| | channel blockade | increase action potential duration and effective |
| | | refractory period. |
| IV | calcium-channel | Block L-type calcium channels; most effective |
| | blockade | at SA and AV nodes; reduce rate and |
| | | conduction. |

The present invention focuses on the first three classes, i.e., class I, class II, and class III anti-arrhythmic medications, as those are the only ones commonly used to prevent SCA. Table 3 shows anti-arrhythmic medications utilized for various arrhythmic conditions.

TABLE 3

| Condition | Drug | Comments |
|---|---|---|
| Sinus tachycardia | Class II, IV | |
| Atrial fibrillation/flutter | Class IA, IC, II, | Ventricular rate control |
| | III, IV | is important goal; |
| | digitalis | anticoagulation |
| | | is required. |
| Paroxysmal | Class IA, IC, II, | |
| supraventricular | III, IV | |
| tachycardia | adenosine | |
| AV block | atropine | Acute reversal |
| Ventricular tachycardia | Class I, II, III | |
| Premature ventricular | Class II, IV | PVCs are often benign |
| complexes | magnesium sulfate | and do not require |
| | | treatment |
| Digitalis toxicity | Class IB | |
| | magnesium sulfate | |

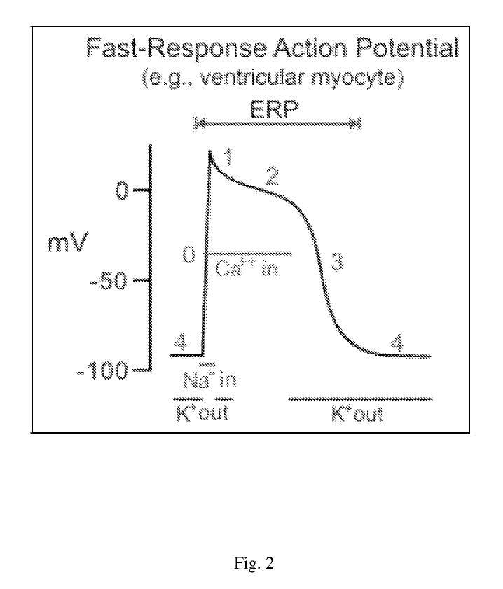

The mechanism of action of the anti-arrhythmic medications as well as the molecular information present in the scientific literature. Na⁺ channel blockers are Type I anti-arrhythmic medications that bind and block the fast sodium channels that are responsible for the rapid depolarization (phase 0) is shown in FIG. 2. They may also alter the action potential duration (“APD”) and the effective refractory period (“ERP”) due to the action of the drug on potassium channels that are involved in phase 3 repolarization of action potentials. The details of genes coding the Na⁺ channels are shown in Table 4 (Molecular Basis of Cardiovascular Disease: A Companion to Braunwald's Heart Disease, Kenneth R. Chien (Author), Saunders;

Revised edition (2003) pp. 315-327).

TABLE 4

| Ion Current | Coding Gene | # of aa | # of SNPs | Illumina Markers |
|---|---|---|---|---|
| I_{Na} | SCN5A | 2015 | 196 | 24 |
| I_{Na} | SCN5A | 218 | 109 | 27 |

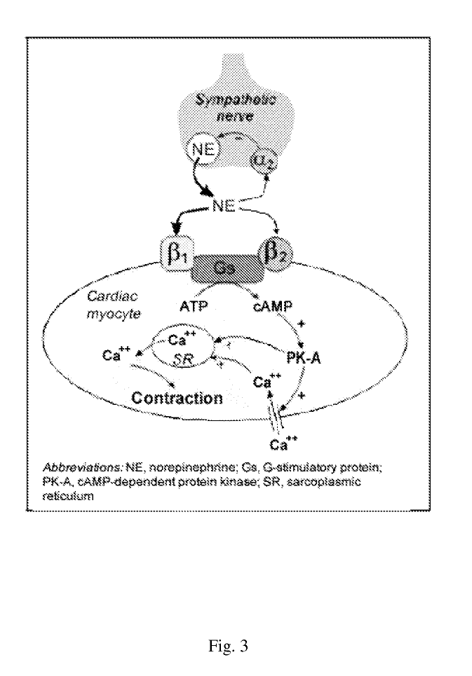

Beta-blockers (“β-blockers”) are Type II anti-arrhythmic medications that bind to beta-adrenoceptors located in cardiac nodal tissue, the conducting system, and contracting myocytes. The heart has both beta-1 (“β1”) and beta-2 (“β2”) adrenoceptors, although the primary receptor type in number and function is β1. These receptors primarily bind norepinephrine that is released from sympathetic adrenergic nerves as shown in FIG. 3. The details of genes coding proteins involved in the actions of the β-blockers are shown in Table 5 (Cardiovascular Genetics and Genomics for the Cardiologist, by Victor J. Dzau MD (Editor) and Choong-Chin Liew PhD (Editor), Wiley-Blackwell, 1st edition (Aug. 3, 2007), p. 260).

TABLE 5

| Ion Current | Coding Gene | # of aa | # of SNPs | Illumina Markers |
|---|---|---|---|---|
| β-B | ADRB1 | 477 | 186 | 21 |
| β-B | ADRB2 | 413 | 239 | 32 |
| β-B | CYP2D6 | 497 | 109 | 10 |

K⁺ channel blockers are Type III anti-arrhythmic medications that bind to and block the K⁺ channels that are responsible for phase 3 repolarization as shown in FIG. 2. Blocking these channels slows and delays repolarization, which leads to an increase in action potential duration (“APD”) and an increase in the effective refractory period (“ERP”). Details of the genes coding the K+channels are shown in Table 6 (Chien).

TABLE 6

| Ion Current | Coding Gene | # of aa | # of SNPs | Illumina Markers |
|---|---|---|---|---|
| I_{tO} | KCNA4 | 653 | 141 | 19 |
| I_{tO} | KCNAB1 | 401 | 153 | 16 |
| I_{tO} | KCND2 | 629 | 222 | 14 |
| I_{tO} | KCNE2 | 123 | 188 | 30 |
| I_{tO} | KCNIP2 | 252 | 56 | 12 |
| I_{tO} | KCND3 | 656 | 363 | 49 |
| I_{Kr} | KCNH2 | 1159 | 114 | 24 |
| I_{Ks} | KCNQ1 | 676 | 162 | 30 |
| I_{Ks} | KCNE1 | 129 | 222 | 43 |
| I_{Kur} | KCNA5 | 611 | 346 | 39 |
| I_{K1} | KCNJ2 | 427 | 278 | 27 |

The number of SNPs shown in Tables 4, 5 and 6, was attained by using the Haploview application(I) (version 4.1). The HapMap Download feature was used to open a new data set. The GeneCruiser feature of Haploview allows for selection of a gene locus by Ensembl ID and a surrounding flanking region that is determined by the user. For the gene of interest, the Ensembl ID was retrieved from the Ensembl Genome Browser by searching on the gene name. The Ensembl ID and a flanking region of 100 kb were then used to determine the chromosomal region from which to identify associated SNPs. This number then represents the number of SNPs within a gene of interest, as indicated in the “# of SNPs” column. For each gene, the associated SNPs were saved into a local file by rs number and queried against the SNPs used in the genotyping assay, which was performed using the HumanHap300 BeadChip, which includes 317,503 tagSNPs selected from the Phase I International HapMap Project. For example, there were 141 SNPs identified within the KCNA4 gene and 19 of these 141 SNPs were on the HumanHap300 BeadChip identified as Illumina markers in the Tables 4, 5 and 6.

An explanation of an rs number and dbSNP is provided herein. In collaboration with the National Human Genome Research Institute, The National Center for Biotechnology Information has established the Single Nucleotide Polymorphism Database (dbSNP) database to serve as a central repository for both single base nucleotide substitutions, also known as single nucleotide polymorphisms (SNP) and short deletion and insertion polymorphisms. Once a new SNP is submitted to dbSNP, it is assigned a unique submitted SNP ID number (ss#). Once the ss number is assigned, the flanking sequence of each submitted SNP is aligned to its appropriate genomic contig. If several ss numbers map to the same position on the contig, they are clustered together into a “reference SNP cluster”, or “refSNP”, and the cluster is assigned a unique RefSNP ID number (rs#). If only one ss number maps to a specific position, then that ss is assigned an rs number and is the only member of its RefSNP cluster unless another submitted SNP is found that maps to the same position. Hence, it is understood that rs numbers can be used to uniquely identify a SNP and fully enables one of ordinary skill in the art to make and use the invention using rs numbers.

To identify genetic markers associated with SCA or SCD, a sub-study (also referred to herein as “MAPP”) to an ongoing clinical trial (also referred to herein as “MASTER”) was designed and implemented. The MASTER study was undertaken to determine the utility of T-wave-alternans test for the prediction of SCA in patients who have had a heart attack and are in heart failure. If necessary to understand the invention, the subject matter of U.S. patent application Ser. No. 12/271,338 is incorporated herein by reference. The data collected from the patients participating in the MAPP study were retrospectively analyzed to search for genetic markers that may be associated with patients being unresponsive to anti-arrhythmic medications. The MAPP study was a prospective study of 240 patients who had an ICD implanted at enrollment, with a 2.6 year mean follow-up period. Thirty-three of the patients experienced life threatening arrhythmias (“LTAs”) and were considered case subjects. The remaining 207 patients did not have LTAs, and hence they were considered control subjects.

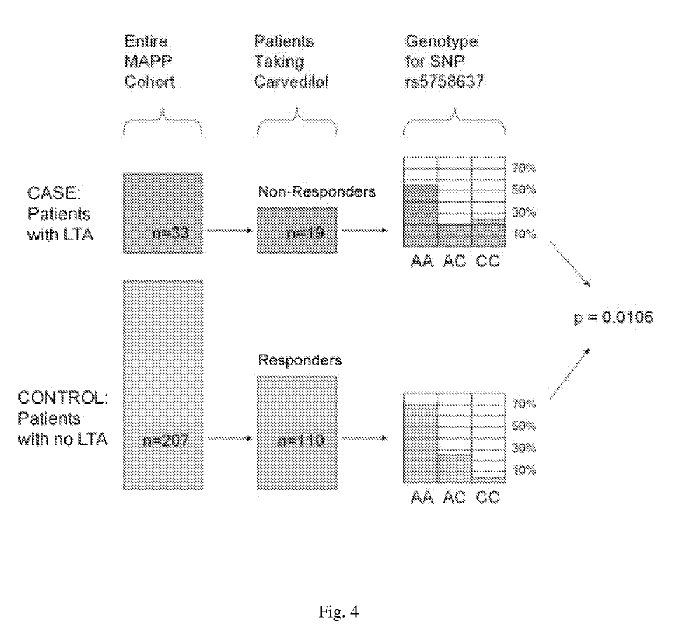

In the MAPP study, the anti-arrhythmic medications taken by the patients were identified from the case report forms (“CRF”). For each medication, a new patient cohort was generated using only the subjects who were taking the given medication. The patients were re-categorized during the follow-up period within this new cohort based on whether a patient experienced an arrhythmic event (Non-Responders) or not (Responders). Only the genetic data containing the SNPs listed in Tables 3, 4, or 5 were then analyzed, according to the anti-arrhythmic drug administered. Finally, the p-values were calculated for any given SNP to determine if that particular SNP could be a marker for a non-response to anti-arrhythmic medication. FIG. 4 illustrates an example of the process.

Typically, association of genetic variation and disease can be a function of many factors, including, but not limited to, the frequency of the risk allele or genotype, the relative risk conferred by the disease-associated allele or genotype, the correlation between the genotyped marker and the risk allele, sample size, disease prevalence, and genetic heterogeneity of the sample population. For each locus, two nucleic acid reads were done from each patient, representing the nucleotide variants on two chromosomes, except for the loci chromosomes on male patients. Four letter symbols were used to represent the nucleotides that were read: cytosine (C), guanine (G), adenine (A), and thymine (T). The structure of the various alleles is described by any one of the nucleotide symbols of Table 7.

TABLE 7
Allele Key used in Sequence Listings

| Nucleotide symbol | Full Name |
|---|---|
| R | Guanine/Adenine (purine) |
| Y | Cytosine/Thymine (pyrimidine) |
| K | Guanine/Thymine |
| M | Adenine/Cytosine |
| S | Guanine/Cytosine |
| W | Adenine/Thymine |
| B | Guanine/Thymine/Cytosine |
| D | Guanine/Adenine/Thymine |
| H | Adenine/Cytosine/Thymine |
| V | Guanine/Cytosine/Adenine |
| N | Adenine/Guanine/Cytosine/Thymine |

Table 8 contains a summary list of the anti-arrhythmic drugs that were studied in the MAPP study.

TABLE 8

| Class I | | |
|---|---|---|
| Na⁺ Channel | Class II | Class III |
| Blockers | Beta-Blockers | K⁺ Channel Blockers |
| Disopyramide | Atenolol | Amiodarone |
| Flecainide | Betaxolol | Dofetilide |
| Moricizine | Bucindolol | Sotalol |
| Procainamide | Carvedilol | |
| Propafenone | Metoprolol | |
| Quinidine | Nadolol | |
| | Penbutolol | |
| | Propranolol | |
| | Timolol | |

The majority of the patients in the MAPP study were taking either Carvedilol or Metoprolol, both of which are beta-blocker medications. This is expected, since β-blockers are commonly used for the prevention of SCA, because they are indicated for this use in the American Heart Association (“AHA”) guidelines. Therefore, statistical analysis was conducted for those two medications by calculating the p-values where the Cochran-Armitage test was used with the contingency table of genotypes derived from the life threatening arrhythmia event status with an assumption that the risk allele has an additive effect. The Cochran-Armitage Test is a test for trend to determine if there is a difference in the dosage effect between two groups. Dosage refers to the number of risk alleles, which is 0, 1 or 2 and the two groups are subjects with (non-responders) or without (responders) life-threatening ventricular tachycardia or fibrillation. Results are shown in Tables 9 and 10 below. The two Genotype Counts columns are triplets indicating the number of subjects with 0, 1 and 2 risk alleles, respectively. For example, for rs5758637, the responder column is 76/30/4 and the non-responder column is 10/4/5. Among the responders, 76 had the AA genotype (0 risk alleles), 30 had the AC genotype (1 risk allele) and 4 had the CC genotype (2 risk alleles). Among the non-responders 10, 4 and 5 subjects had the AA, AC and CC genotypes, respectively. Specifically, Table 8 shows the SNPs that were tested for predicting patient response to Carvedilol.

TABLE 9

| | | | | | Genotype Counts | |
|---|---|---|---|---|---|---|
| SNP | p-value | Gene | Nucleotides | Risk Allele | Resp | Non-Resp |
| rs5758637 | 0.010615 | CYP2D6 | A/C | C | 76/30/4 | 10/4/5 |
| rs3857420 | 0.034935 | ADRB2 | C/T | C | 66/38/7 | 16/3/0 |
| rs5758651 | 0.043806 | CYP2D6 | T/C | C | 82/24/4 | 11/5/3 |
| rs7894582 | 0.099561 | ADRB1 | C/A | A | 14/96/0 | 0/19/0 |
| rs6888011 | 0.104549 | ADRB2 | T/C | T | 51/52/8 | 12/7/0 |
| rs742086 | 0.109149 | CYP2D6 | T/G | G | 67/39/5 | 9/7/3 |
| rs919725 | 0.115906 | ADRB2 | C/A | C | 51/43/17 | 11/8/0 |
| rs888956 | 0.165565 | ADRB2 | A/C | A | 65/39/6 | 14/5/0 |
| rs151591 | 0.172768 | ADRB1 | G/A | A | 65/39/7 | 7/11/1 |
| rs11957757 | 0.279701 | ADRB2 | G/A | G | 36/53/22 | 8/9/2 |
| rs10885522 | 0.311772 | ADRB1 | G/A | A | 15/96/0 | 1/18/0 |
| rs12654778 | 0.313545 | ADRB2 | G/A | A | 42/51/17 | 5/10/4 |
| rs1864932 | 0.315974 | ADRB2 | A/G | A | 31/52/28 | 8/7/4 |
| rs1042713 | 0.317218 | ADRB2 | G/A | A | 42/52/17 | 5/10/4 |
| rs11168074 | 0.324144 | ADRB2 | T/C | T | 50/45/16 | 9/10/0 |
| rs741146 | 0.329182 | ADRB2 | G/T | T | 45/56/10 | 7/8/4 |
| rs4705280 | 0.348457 | ADRB2 | G/T | G | 37/52/19 | 8/8/2 |
| rs1034258 | 0.35181 | ADRB1 | T/C | T | 57/45/9 | 12/6/1 |
| rs12484402 | 0.362034 | CYP2D6 | C/T | T | 60/38/13 | 9/6/4 |
| rs2400642 | 0.371716 | ADRB2 | A/G | A | 67/39/5 | 13/6/0 |
| rs10515621 | 0.38757 | ADRB2 | T/C | C | 76/31/4 | 12/5/2 |
| rs2480792 | 0.416703 | ADRB1 | G/A | G | 38/55/18 | 7/11/1 |
| rs740746 | 0.419696 | ADRB1 | A/G | A | 63/40/7 | 13/5/1 |
| rs5996130 | 0.444842 | CYP2D6 | G/A | G | 92/18/1 | 17/2/0 |
| rs30325 | 0.446124 | ADRB2 | A/G | A | 37/51/23 | 8/8/3 |
| rs877741 | 0.446755 | ADRB2 | T/C | T | 71/32/8 | 14/4/1 |
| rs82625 | 0.456377 | ADRB1 | G/A | A | 12/99/0 | 1/18/0 |
| rs1801311 | 0.482373 | CYP2D6 | C/T | T | 47/47/16 | 7/8/4 |
| rs11090076 | 0.489658 | CYP2D6 | T/C | C | 47/48/16 | 7/8/4 |
| rs2413669 | 0.489658 | CYP2D6 | A/C | C | 47/48/16 | 7/8/4 |
| rs764481 | 0.489658 | CYP2D6 | G/A | A | 47/48/16 | 7/8/4 |
| rs9325124 | 0.491394 | ADRB2 | G/A | G | 44/44/23 | 8/9/2 |
| rs6884617 | 0.510447 | ADRB2 | T/C | C | 33/47/31 | 4/9/6 |
| rs1181141 | 0.582851 | ADRB2 | T/G | G | 75/35/1 | 16/1/2 |
| rs4359161 | 0.623464 | ADRB1 | G/A | G | 71/38/2 | 13/6/0 |
| rs30306 | 0.626743 | ADRB2 | A/G | A | 29/55/27 | 7/7/5 |
| rs180925 | 0.669887 | ADRB1 | C/A | C | 63/34/14 | 8/10/1 |
| rs11959113 | 0.678142 | ADRB2 | G/A | G | 63/39/9 | 9/9/1 |
| rs9285673 | 0.683327 | ADRB2 | A/C | C | 85/23/3 | 16/2/1 |
| rs10490907 | 0.705858 | ADRB1 | A/C | A | 94/15/2 | 15/4/0 |
| rs2142695 | 0.726359 | CYP2D6 | C/T | T | 102/9/0 | 17/2/0 |
| rs3813720 | 0.731279 | ADRB1 | T/C | C | 44/58/9 | 9/8/2 |
| rs426121 | 0.736354 | ADRB1 | G/A | G | 99/10/2 | 16/3/0 |
| rs6585258 | 0.752954 | ADRB1 | G/T | T | 40/49/22 | 6/9/4 |
| rs1411407 | 0.764283 | ADRB1 | C/T | C | 30/57/24 | 5/11/3 |
| rs180950 | 0.814442 | ADRB1 | T/G | T | 41/51/19 | 5/12/2 |
| rs4705284 | 0.827883 | ADRB2 | C/T | C | 86/24/1 | 15/4/0 |
| rs10885531 | 0.830177 | ADRB1 | T/C | T | 23/67/21 | 6/8/5 |
| rs6580586 | 0.849851 | ADRB2 | A/C | A | 90/16/5 | 15/4/0 |
| rs151603 | 0.859129 | ADRB1 | A/G | A | 42/48/21 | 5/12/2 |
| rs4705286 | 0.890041 | ADRB2 | T/C | T | 65/37/9 | 11/7/1 |
| rs17108911 | 0.89089 | ADRB2 | T/C | T | 5/105/0 | 1/18/0 |
| rs151600 | 0.895173 | ADRB1 | G/A | G | 43/44/23 | 5/12/2 |
| rs1042718 | 0.901024 | ADRB2 | C/A | A | 70/36/3 | 13/5/1 |
| rs2050394 | 0.97756 | ADRB1 | A/G | A | 85/20/1 | 15/4/0 |
| rs10490905 | 0.987119 | ADRB1 | T/C | T | 84/25/2 | 14/5/0 |
| rs11742884 | 0.994892 | ADRB2 | T/C | T | 77/27/7 | 13/5/1 |

Table 10 shows the SNPs that were tested for predicting patient response to Metoprolol.

TABLE 10

| | | | | | Genotype Counts | |
|---|---|---|---|---|---|---|
| SNP | p-value | Gene | Nucleotides | Risk Allele | Resp | Non-Resp |
| rs151603 | 0.001269 | ADRB1 | A/G | G | 22/29/9 | 0/2/5 |
| rs151600 | 0.002008 | ADRB1 | G/A | A | 21/29/10 | 0/2/5 |
| rs180925 | 0.024782 | ADRB1 | C/A | A | 30/26/4 | 1/4/2 |
| rs11957757 | 0.024916 | ADRB2 | G/A | A | 17/30/13 | 0/3/4 |
| rs2082382 | 0.029146 | ADRB2 | A/G | G | 21/29/10 | 0/4/3 |
| rs180950 | 0.033094 | ADRB1 | T/G | G | 23/27/10 | 2/0/5 |
| rs151591 | 0.041384 | ADRB1 | G/A | A | 36/20/4 | 2/3/2 |
| rs7711757 | 0.062742 | ADRB2 | T/C | T | 38/20/2 | 7/0/0 |
| rs741146 | 0.070093 | ADRB2 | G/T | G | 22/30/8 | 5/2/0 |
| rs6580586 | 0.107656 | ADRB2 | A/C | A | 42/16/2 | 7/0/0 |
| rs758586 | 0.123312 | ADRB1 | A/G | G | 27/24/9 | 1/4/2 |
| rs7894582 | 0.213324 | ADRB1 | C/A | C | 54/4/1 | 5/2/0 |
| rs1181141 | 0.214538 | ADRB2 | T/G | T | 7/53/0 | 2/5/0 |
| rs1042718 | 0.229115 | ADRB2 | C/A | C | 39/14/7 | 6/1/0 |
| rs9285673 | 0.233794 | ADRB2 | A/C | A | 49/10/1 | 7/0/0 |
| rs12484402 | 0.308329 | CYP2D6 | C/T | C | 27/29/4 | 1/6/0 |
| rs426121 | 0.325888 | ADRB1 | G/A | G | 52/7/1 | 7/0/0 |
| rs2480792 | 0.33359 | ADRB1 | G/A | A | 20/28/12 | 1/4/2 |
| rs82625 | 0.335347 | ADRB1 | G/A | A | 52/8/0 | 6/0/1 |
| rs4359161 | 0.335347 | ADRB1 | G/A | G | 41/18/1 | 6/1/0 |
| rs1042713 | 0.351269 | ADRB2 | G/A | G | 26/28/6 | 4/3/0 |
| rs11742884 | 0.377351 | ADRB2 | T/C | T | 35/19/6 | 5/2/0 |
| rs17108911 | 0.427217 | ADRB2 | T/C | C | 5/55/0 | 0/7/0 |
| rs1034258 | 0.464498 | ADRB1 | T/C | C | 37/17/6 | 6/0/1 |
| rs12654778 | 0.467262 | ADRB2 | G/A | G | 29/25/6 | 4/3/0 |
| rs11090076 | 0.481132 | CYP2D6 | T/C | C | 23/26/11 | 1/5/1 |
| rs2142695 | 0.481132 | CYP2D6 | C/T | C | 56/4/0 | 7/0/0 |
| rs2413669 | 0.481132 | CYP2D6 | A/C | C | 23/26/11 | 1/5/1 |
| rs764481 | 0.481132 | CYP2D6 | G/A | A | 23/26/11 | 1/5/1 |
| rs1411407 | 0.486995 | ADRB1 | C/T | T | 14/34/12 | 0/6/1 |
| rs11168074 | 0.512111 | ADRB2 | T/C | T | 24/27/9 | 3/4/0 |
| rs1801311 | 0.514054 | CYP2D6 | C/T | T | 23/26/11 | 1/4/1 |
| rs10885531 | 0.522351 | ADRB1 | C/T | T | 19/32/9 | 1/5/1 |
| rs6884617 | 0.536311 | ADRB2 | T/C | C | 20/30/10 | 1/5/1 |
| rs6888011 | 0.577667 | ADRB2 | T/C | T | 33/19/8 | 4/3/0 |
| rs742086 | 0.631949 | CYP2D6 | T/G | T | 40/16/4 | 5/2/0 |
| rs1864932 | 0.639143 | ADRB2 | G/A | G | 18/24/18 | 2/4/1 |
| rs6585258 | 0.643864 | ADRB1 | G/T | G | 19/32/9 | 2/5/0 |
| rs2050394 | 0.64944 | ADRB1 | A/G | A | 47/13/0 | 6/1/0 |
| rs4705280 | 0.66743 | ADRB2 | T/G | T | 16/26/15 | 2/4/1 |
| rs10490905 | 0.675703 | ADRB1 | T/C | T | 48/11/1 | 6/1/0 |
| rs5996130 | 0.675703 | CYP2D6 | G/A | G | 48/11/1 | 6/1/0 |
| rs3857420 | 0.707395 | ADRB2 | C/T | C | 36/16/8 | 4/3/0 |
| rs10885522 | 0.71751 | ADRB1 | G/A | A | 12/48/0 | 1/6/0 |
| rs2400709 | 0.730739 | ADRB2 | G/A | A | 1/59/0 | 0/7/0 |
| rs888956 | 0.736318 | ADRB2 | A/C | A | 35/19/6 | 4/3/0 |
| rs30325 | 0.743278 | ADRB2 | G/A | G | 17/29/14 | 2/4/1 |
| rs10515621 | 0.775579 | ADRB2 | T/C | T | 47/12/1 | 5/2/0 |
| rs919725 | 0.798014 | ADRB2 | C/A | C | 30/20/9 | 3/4/0 |
| rs11959113 | 0.837973 | ADRB2 | G/A | G | 24/32/4 | 2/5/0 |
| rs740746 | 0.860362 | ADRB1 | A/G | G | 27/26/7 | 3/3/1 |
| rs10490907 | 0.872153 | ADRB1 | A/C | A | 50/10/0 | 6/1/0 |
| rs3813720 | 0.884046 | ADRB1 | T/C | T | 20/26/14 | 2/4/1 |
| rs5758651 | 0.943067 | CYP2D6 | T/C | T | 45/12/2 | 5/2/0 |
| rs5758637 | 0.947842 | CYP2D6 | A/C | A | 45/12/3 | 5/2/0 |
| rs4705286 | 0.963419 | ADRB2 | T/C | T | 31/23/6 | 3/4/0 |
| rs30306 | 0.980671 | ADRB2 | G/A | G | 22/25/13 | 2/4/1 |
| rs2400642 | 0.983567 | ADRB2 | A/G | G | 36/22/2 | 5/1/1 |

For the overall analysis, a total of 63 SNPs were tested. To reach statistical significance, it is desirable to have p-values less

$$\frac{0.05}{63} \approx 0.00079 ,$$

than according to Bonferroni's rule for multiple comparisons. Although no SNPs reached that level of significance, the top SNPs by p-value are shown in Table 11, indicating that several SNPs were close to being statistically significant for predicting patient response to anti-arrhythmic medications.

TABLE 11

| Anti- | | | | | Risk |
|---|---|---|---|---|---|
| Arrhythmic | SNP | p-value | Gene | Nucleotides | Allele |
| Carvedilol | rs5758637 | 0.010615 | CYP2D6 | A/C | C |
| Metoprolol | rs151603 | 0.001269 | ADRB1 | A/G | G |
| Metoprolol | rs151600 | 0.002008 | ADRB1 | G/A | A |

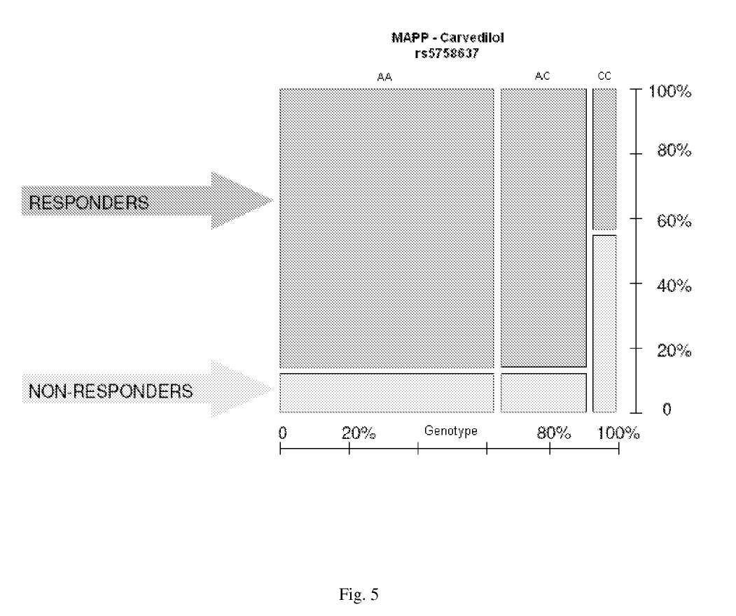

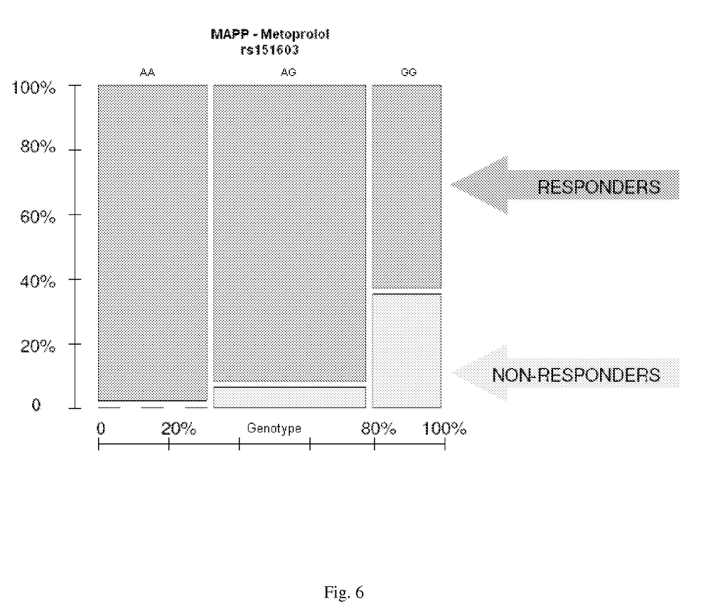

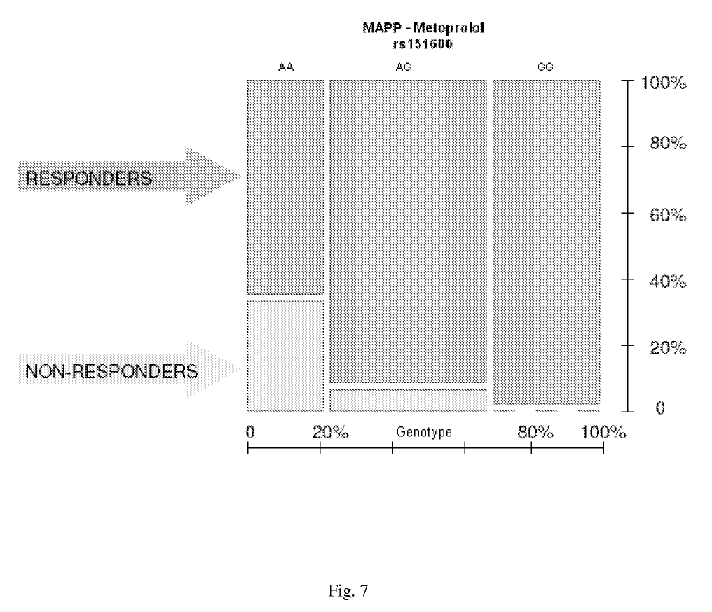

Additional analysis was conducted to identify other SNPs that can be surrogates to the SNPs shown in Table 11. This analysis was accomplished by searching for SNPs that are in the same haplotype region as the SNPs in Table 11. With regard to the identification of risk alleles for surrogate SNPs, it is noted that surrogate SNPs were designated as such because they were in haplotype blocks with an initial SNP of interest. Haplotype blocks result from linkage disequilibrium, which is the process wherein ranges of nucleotides are inherited together more than expected. During DNA replication, the two DNA strands experience crossover events. If these events were entirely at random, it would be difficult to predict SNPs from neighboring SNPs. Some regions tend to be resistant to crossover events, leading to linkage disequilibrium and thereby haplotype blocks, which make it more reasonable to predict SNPs from neighboring SNPs. Because SNPs within haplotype blocks tend to be inherited together, the relative frequencies of the minor alleles should be similar. Thus, if the risk allele for a SNP of interest is the minor allele, the risk allele of a SNP within the haplotype block will also tend to be the minor allele. Moreover, haplotype regions of the genome are the segments that are inherited together, along with the SNPs within that region. Hence, once the nucleotide in a SNP is known, one of ordinary skill can infer the genotype of the remaining nucleotides within the same haplotype region. Therefore, SNPs located within the same haplotype region of a marker SNP can serve as surrogate markers wherein a risk allele for the surrogate marker can be determined using statistical correlations. For example, one of ordinary skill could assume that a SNP1 and SNP2 are in the same haplotype region, and where it was determined with HapMap analysis that when a patient has the nucleotide “A” in the SNP1 location, the patient has a “G” at the SNP2 location. On the other hand, when a patient has a “C” at the SNP1 location, then the patient has a “T” at the SNP2 location. When clinical analysis determines that the SNP1 is the risk marker, and the risk allele is “C”, then the SNP2 could be a surrogate marker with the risk allele of “T”. This analysis applies to all the surrogate markers on the present invention. Table 12 shows the results of such an analysis wherein each of the risk allele for each of the surrogate SNPs can be determined according to the statistical study described herein. FIGS. 5, 6, and 7 contain the mosaic plots for the SNPs listed in Table 11.

TABLE 12

| Anti- | | |
|---|---|---|
| Arrhythmic | | |
| Medication | SNP | Surrogate SNPs |
| Carvedilol | rs5758637 | rs5758627, rs9607885, rs2142695, |
| | | rs17002868, rs12484402, rs5751239, |
| | | rs5751240, rs9623538, rs17002872, |
| | | rs5758645, rs17002876 |
| Metoprolol | rs151603 | rs151591, rs151594, rs151595, rs6585252, |
| | | rs11196566, rs151599, rs151600, rs7099933, |
| | | rs151602, rs151603, rs180935, rs180934, |
| | | rs180932, rs180929, rs7077623, rs180928, |
| | | rs11196573, |
| Metoprolol | rs151600 | rs11196575, rs180925, rs180923, rs180922, |
| | | rs180921, rs1860398, rs180919, rs180918, |
| | | rs180917, rs180915, rs180914, rs17653278, |
| | | rs180913, rs17574901, rs180912, rs180910, |
| | | rs180909, rs180908 |

The knowledge of the 101 base sequence, including the SNP of interest, the 50 base nucleotide sequence prior to (5′) and subsequent to (3′) the SNP, along with the chromosome number and the chromosome band can be sufficient to unambiguously identify a SNP in the human genome.

One embodiment involves the screening of patients through a clinical utilization of a genetic test to determine the patients' susceptibility to life threatening arrhythmias. To determine a patient's risk of SCA, or SCD, the following screening process is performed: A genetic information source, such as a biological sample or sequence data, is collected. Biological samples can be of any type such as tissue, blood, etc. Genetic information may also be obtained from sequence data in the form of electronic, print, or any other recorded media or from a SNP read from the patient. If the genetic information is to be extracted from tissue samples, a genetic information extraction system is used. This system may be implemented in steps. First, DNA is extracted. DNA extraction may be performed using any of a number of techniques using any of a number of techniques including phenol-chloroform extraction, phenol-chloroform extraction followed by ethanol precipitation or isopropanol precipitation of DNA, glass bead purification, or salt precipitation. (See Current Protocols in Molecular Biology, Pub. by John Wiley & Sons, updated annually; see Miller, S. A. et al., “A simple salting out procedure for extracting DNA from human nucleated cells,” Nucleic Acids Res., 1988; 16 (3): 1215). DNA extraction kits from commercial vendors such as Qiagen and Stratagene may also be used. Second, the genetic sequence is read using any of the following techniques: (1) sequencing, such as Sanger sequencing technique following PCR; (2) DNA microarray chips, SNP Chips, or genetic data services; (3) SNP Stream; (4) bead arrays, e.g., AmpaSand SIFT; (5) mass spectrometry (sequenome); (6) fragment analysis using Capillary Electrophoresis; or (7) Taqman Allelic Discrimination Assay. Finally, a computer algorithm or manual chart or table can be used to determine a patient's risk for SCA.

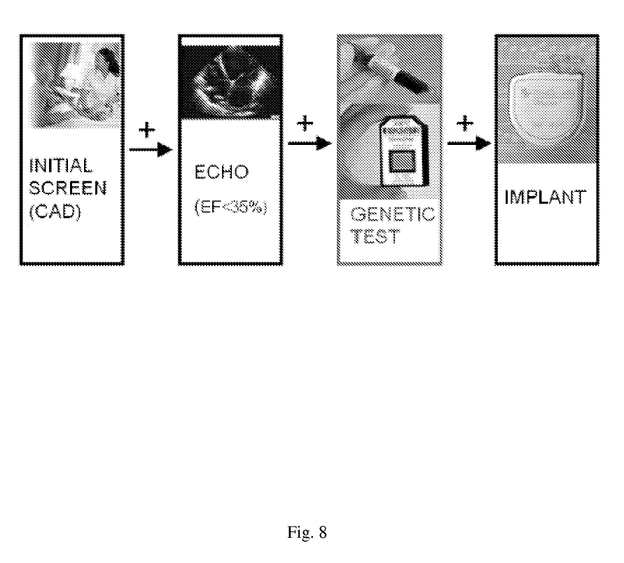

FIG. 8 depicts one embodiment of a clinical utilization of a diagnostic kit involving genetic tests created for screening patients for susceptibility to life threatening arrhythmias based on a non-response to anti-arrhythmic medications. In this embodiment, patients already testing positively for CAD and a low EF would undergo the test for genetic susceptibility using any of the methods described herein. Positive genetic test results would then be used in conjunction with the other test, such as the ones based on the analysis of ECG, and be used to make the ultimate decision of whether or not to implant an ICD. Patients who are already presenting a cardiac condition such as myocardial infarction (“MI”) are usually subjected to echocardiographic examination to determine the need for an ICD. Based on the present invention, blood samples could also be taken from the patients who have low left ventricular EF. If the diagnostic kit indicates that the patient could not be sufficiently protected using beta-blocker therapy, then a recommendation is made for an ICD implant. A schematic of this overall process is shown in FIG. 8.

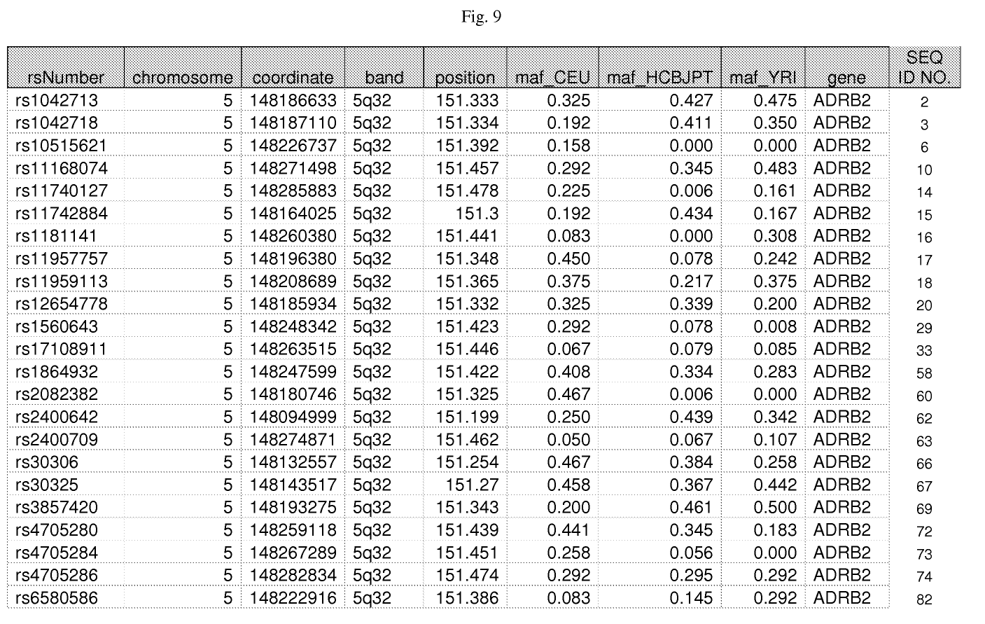

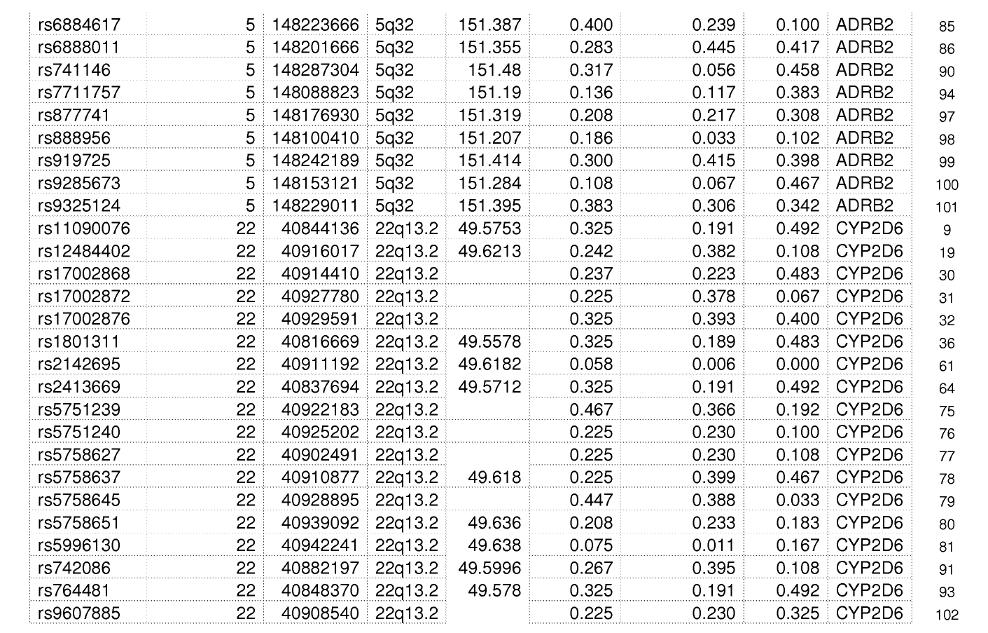

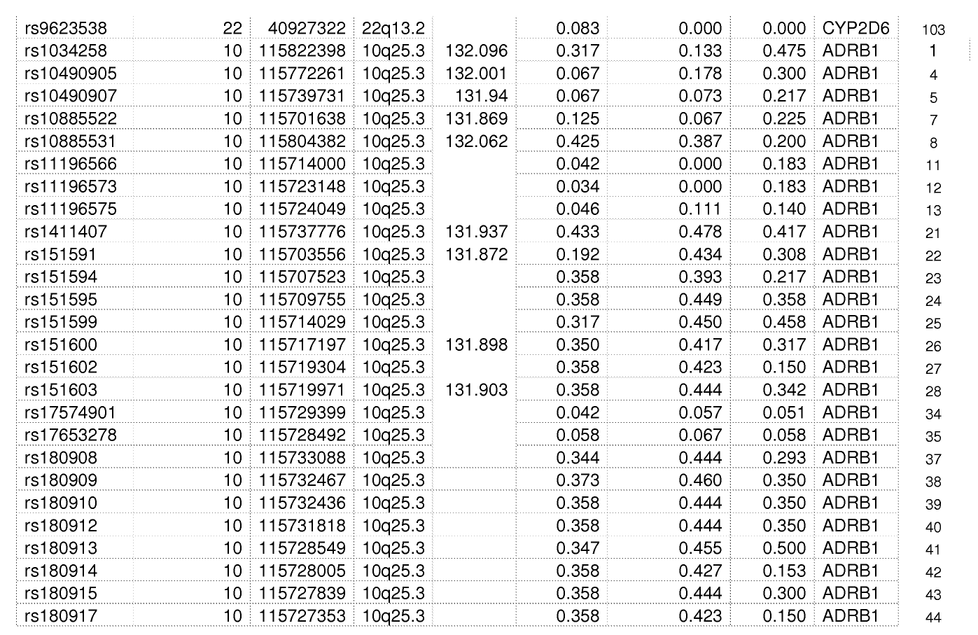

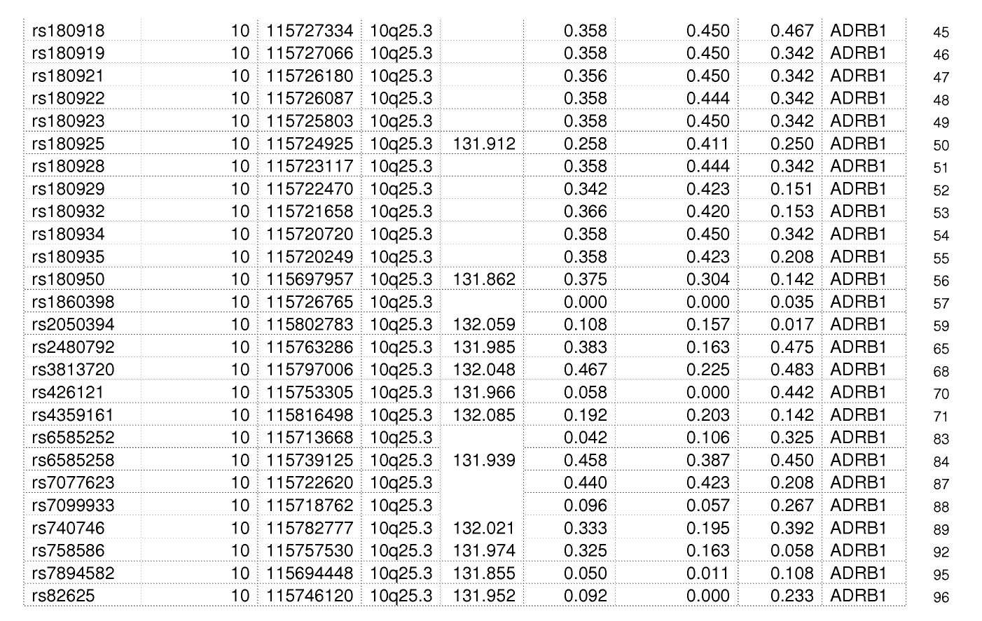

FIG. 9 provides the chromosome, coordinate band, position maf_CEU maf_HCBJPT, maf_YRIF wherein the three maf fields indicate the minor allele frequency within European-descent, Asian and African populations, respectively. This information can be correlated with a European patient population as provided herein.

### EXAMPLES

### Bead-based Genotyping and Haplotyping

A template can be generated by obtaining genomic DNA probes representing the SNPs of SEQ ID NO.'s 11-13, 19, 22-28, 30-32, 34-35, 37-55, 57, 61, 75-79, 83-88 and 102-103. Nested PCR can be used to generate a template for typing where amplifications could be performed using PCR Mastermix (Abgene, Inc., Rochester, N.Y.). Primary PCRs can be carried out with 20 ng genomic DNA in 10 μl 1×PCR Mastermix, 0.2 μM of primers, and 2 mM MgCl₂ with the following cycling conditions: 95° C. for 5 min; 40 cycles at 95° C. for 30 s, 58° C. for 30 s, 72° C. for 2 min 30 s; 72° C. for 10 min. The product can then be diluted 1:500 in 1×TE and re-amplified using asymmetric PCR. The amplified products can then be analyzed by gel electrophoresis and then used directly in a bead-based genotyping and haplotyping reaction.

### Allele-specific Hybridization

For genotyping and haplotyping, allele-specific oligonucleotides (ASOs), representing the SNPs of SEQ ID NO.'s 11-13, 19, 22-28, 30-32, 34-35, 37-55, 57, 61, 75-79, 83-88 and 102-103 can be synthesized. The ASO can be 25 nucleotides long with a 5′ Uni-Link amino modifier where each ASO can be attached to a different colored bead. Genotyping can be performed in a 30 μl hybridization reaction containing 5 μl unpurified PCR product, 83 nM biotinylated sequence-specific oligonucleotide and beads corresponding to each allele of the SNPs of SEQ ID NO.'s 11-13, 19, 22-28, 30-32, 34-35, 37-55, 57, 61, 75-79, 83-88 and 102-103 reacted in lx TMAC buffer (4.5 M TMAC, 0.15% Sarkosyl, 75 mM Tris-HCl, pH 8.0 and 6 mM EDTA, pH 8.0). The reactions can then be denatured at 95° C. for 2 min and incubated at 54° C. for 30 min. An equal volume of 20 μg/ml streptavidin-R-phycoerythrin (RPE) (Molecular Probes, Inc., Eugene, Oreg.) in 1×TMAC buffer can be added and the reaction be incubated at 54° C. for 20 min prior to analysis on a Luminex 100. The data collection software can be set to analyze 100 beads from each set and the median relative fluorescent intensity can be used for analysis. Visual genotypes and haplotypes can be generated using the online software applications found at http://pga.gs.washington.edu/software.html.

### Genetic Information Extraction System

A genetic information source, such as a biological sample, or sequence data from tissue samples can be of any type such as blood, skin, etc is gathered. Genetic information may also be obtained from sequence data in the form of electronic, print, or any other recorded media or from a SNP read from the patient. A genetic information extraction system (if the information is to be extracted from a tissue sample). This can be done in the following steps:

(a) Extract DNA

DNA extraction may be performed using any of a number of techniques including phenol-chloroform extraction, phenol-chloroform extraction followed by ethanol precipitation or isopropanol precipitation of DNA, glass bead purification, or salt precipitation (See Current Protocols in Molecular Biology, Published by John Wiley & Sons, updated annually and Miller, S. A., Dykes, D. D. and Polesky, H. F. (1988), Nucleic Acids Res 16 (3):1215). DNA extraction kits from commercial vendors such as Qiagen and Stratagene may also be used.

(b) Read Genetic Sequence

Genetic sequence can be read using any of the following techniques: Sequencing, such as Sanger sequencing technique following PCR, DNA microarray chips/SNP Chips/Genetic data services, SNP Stream, Bead arrays (e.g. AmpaSand SIFT), Mass spectrometry (sequenome), Fragment Analysis using Capillary Electrophoresis, Taqman Allelic Discrimination Assay. A computer algorithm can be used to determine the patient's risk for SCD.

It should be understood that the above-described embodiments and examples are merely illustrative of some of the many specific embodiments that represent the principles of the present invention. Clearly, numerous other versions can be readily devised by those skilled in the art without departing from the scope of the present invention.

1. (canceled) An isolated nucleic acid molecule having a Single Nucleotide Polymorphism (SNP) at position 51 selected from the group consisting of SEQ ID NO.'s 11-13, 19, 22-28, 30-32, 34-35, 37-55, 57, 61, 75-79, 83-88 and 102-103, or a complement thereof.

2. (canceled)

3. (canceled)

4. (canceled)

5. (canceled)

6. (canceled)

7. (canceled)

8. (canceled)

9. (canceled)

10. (canceled)

11. (canceled

12. (canceled)

13. (canceled)

14. (canceled)

15. (canceled)

16. A diagnostic kit for detecting one or more polymorphisms in a genetic sample from a human subject refractory to Carvedilol comprising, at least one probe for assessing the presence of a Single Nucleotide Polymorphism (SNP) at position 51 in any one of SEQ ID NO.'s 78, 77, 102, 61, 30, 19, 75, 76, 103, 31, 79, and 32.

17. The diagnostic kit of claim 16, wherein if a risk allele C is detected at position 51 in SEQ ID NO. 78, then the implantation of an Implantable Cardiac Defibrillator (ICD) is recommended in the human subject.

18. A diagnostic kit for detecting one or more polymorphisms in a genetic sample from a human subject refractory to Metoprolol comprising, at least one probe for assessing the presence of a Single Nucleotide Polymorphism (SNP) at position 51 in any one of SEQ ID NO.'s 28, 22, 23, 24, 83, 11, 25, 26, 88, 27, 28, 55, 54, 53, 52, 87, 51, 12, 13, 50, 49, 48, 47, 57, 46, 45, 44, 43, 42, 35, 41, 34, 40, 39, 38 and 37.

19. The diagnostic kit of claim 18, wherein if a risk allele G is detected at position 51 in SEQ ID NO. 28, then the implantation of an Implantable Cardiac Defibrillator (ICD) is recommended in the human subject.

20. A diagnostic kit for detecting one or more polymorphisms in a genetic sample from a human subject refractory to Metoprolol comprising, at least one probe for assessing the presence of a Single Nucleotide Polymorphism (SNP) at position 51 in any one of SEQ ID NO.'s 26, 22, 23, 24, 83, 11, 25, 26, 88, 27, 28, 55, 54, 53, 52, 87, 51, 12, 13, 50, 49, 48, 47, 57, 46, 45, 44, 43, 42, 35, 41, 34, 40, 39, 38 and 37.

21. The diagnostic kit of claim 20, wherein if a risk allele A is detected at position 51 in SEQ ID NO. 26, then the implantation of an Implantable Cardiac Defibrillator (ICD) is recommended in the human subject.

22. The diagnostic kit in any one of claims 16, 18, and 20, said at least one probe ranging from about 3 base pairs at positions 50 to 52 wherein position 51 is flanked on either the 5′ and 3′ side by a number of base pairs flanking the 5′ and 3′ side of position 51 sufficient to identify the SNP or result in a hybridization.

23. The diagnostic kit in any one of claims 16, 18, and 20, said at least one probe being from 3 to 101 nucleotides in length.

24. The diagnostic kit in any one of claims 16, 18, and 20, said at least one probe being a length selected from the group of from about 5 to 101, from about 7 to 101, from about 9 to 101, from about 15 to 101, from about 20 to 101, from about 25 to 101, from about 30 to 101, from about 40 to 101, from about 50 to 101, from about 60 to 101, from about 70 to 101, from about 80 to 101, from about 90 to 101, and from about 99 to 101 nucleotides in length.

25. The diagnostic kit in any one of claims 16, 18, and 20, further comprising a Polymerase Chain Reaction (PCR) primer set for amplifying nucleic acid fragments corresponding to said at least one probe.

26. The diagnostic kit in any one of claims 16, 18, and 20, wherein said at least one probe has a label capable of being detected.

27. The diagnostic kit of claim 26, wherein the label is detected by electrical, fluorescent or radioactive means.

28. The diagnostic kit in any one of claims 16, 18, and 20, wherein said at least one probe is affixed to a substrate.

29. The diagnostic kit in any one of claims 16, 18, and 20, further comprising computer software to analyze information of a hybridization of said at least one probe in the diagnostic kit.

30. The diagnostic kit in any one of claims 16, 18, and 20, wherein said at least one probe is an Allele Specific Oligomer (ASO).

31. The diagnostic kit in any one of claims 16, 18, and 20, wherein the SNP is bi-allelic.

32. The diagnostic kit in any one of claims 16, 18, and 20, wherein the SNP is multi-allelic.

33. The diagnostic kit in any one of claims 16, 18, and 20, wherein said at least one probe is selected from the group of sense, anti-sense, and naturally occurring mutants, of said at least one probe.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)
